(19) United States
(12) Patent Application Publication
(10) Pub. No.: US 2024/0252119 A1
(43) Pub. Date: Aug. 1, 2024

(54) METHOD FOR NON-INVASIVELY DETERMINING AT LEAST ONE BLOOD PRESSURE VALUE, MEASUREMENT APPARATUS AND SYSTEM FOR DETERMINING BLOOD PRESSURE NON-INVASIVELY

(71) Applicant: PHILIPS MEDIZIN SYSTEME BÖBLINGEN GMBH, Böblingen (DE)

(72) Inventors: Ulrich Joachim PFEIFFER, Boeblingen (DE); Stephan REGH, Boeblingen (DE); Benjamin STOLZE, Boeblingen (DE); Josef BRIEGEL, Eindhoven (NL)

(21) Appl. No.: 16/614,166

(22) PCT Filed: May 16, 2018
(86) PCT No.: PCT/EP2018/062736

§ 371 Date: May 2, 2023

(30) Foreign Application Priority Data
May 17, 2017 (DE) 10 2017 110 770.3

(51) Int. Cl. A61B 5/00; A61B 5/022

(52) U.S. Cl. CPC A61B 5/7242; A61B 5/02225; A61B 5/7239

(57) ABSTRACT

A method for non-invasively determining at least one blood pressure value (SAP1ni, MAP1ni, DAP1ni) from a tissue pressure signal (TP) by means of a pressure cuff (10) applied to an individual is specified, wherein the tissue pressure signal (TP) has a sequence of tissue pressure pulse curves (PKi), the method comprising: identifying (S120) at least two individual tissue pressure pulse curves (PKi) in the tissue pressure signal (TP); determining (S150) at least one amplitude parameter (TPP) and an area parameter (TPA) for each identified tissue pressure pulse curve (PKi), wherein the amplitude parameter (TPP) indicates the amplitude of the identified tissue pressure pulse curve (PKi) and the area parameter (TPA) at least indicates one partial area (TPA.top) enclosed by the tissue pressure pulse curve (PKi); for each identified tissue pressure pulse curve (PKi), determining (S160) a pulsation power parameter (TPWP), describing the shape of the tissue pressure pulse curve (PKi), on the basis of at least the amplitude parameter (TPP) and the area parameter (TPA); producing (S170) a parameter function (TPW-curve), which describes a functional relationship between the determined pulsation power parameters (TPWP) of the tissue pressure pulse curves (PKi) and the assigned clamping pressures (TPc1) at the pressure cuff (10) or measurement times (t); establishing (S180-S195) at least one blood pressure value (SAP1ni, MAP1ni, DAP1ni) on the basis of the parameter function (TPW-curve).

The invention relates to a method for non-invasively determining at least one blood pressure value. It further relates to a measuring device and a system for determining at least one blood pressure value.

To measure blood pressures, an invasive or non-invasive measurement method can be used. In a non-invasive blood pressure measurement method, the arterial pressure is measured by means of a blood pressure monitor on one extremity, usually on the arm. To this end, an air-filled pressure cuff is applied to, for example, an upper arm of an individual, preferably a patient. Then, the pressure cuff is provided with a clamping pressure, which acts on the tissue, so that a pressure change in the vessels of the individual can be detected. The clamping pressure, which is provided to the pressure cuff, is usually changed from a high clamping pressure to a low clamping pressure or from a low clamping pressure to a high clamping pressure. In this type of measurement, an oscillation pressure signal resulting from tissue pressure signals can be detected, which shows a sequence of pressure oscillations. Depending on the pressure curve, an increase or decrease of the oscillation pressure signal can be seen based on an increasing or decreasing clamping pressure, respectively.

The pressure cuff is filled with air and placed around a limb of a patient and provided with increasing or decreasing pressure to detect the blood pressure or pulse fluctuations in the blood pressure at the tissue, wherein amplitudes of the individual oscillation pressure signals are analyzed to determine the systolic and/or diastolic blood pressure value. The pressure cuff can also be referred to as a blood pressure cuff.

The detection of non-invasive blood pressure values requires a well-functioning measuring apparatus which, under different measuring environments, detects the oscillation pressure signals in such a way that a reliable detection of the amplitude values is made possible in order to precisely classify the required blood pressure values. Since the tissue strength and composition between the pressure cuff and the artery, the arterial diameter, the arterial stiffness and the blood pressure differ patient to patient, the measurable amplitude values are different, too. In addition, the pressure cuff must be kept at heart level during the measurement. That means, the detected oscillation pressure signals may look different depending on the measurement environment and the patient's blood pressure. For a usable non-invasive blood pressure measurement, the detected oscillation pressure signal must also have a sufficient signal strength. With conventional pressure cuffs, only pressure oscillations with 90-96% loss of the actually hydraulically transcutaneous detectable tissue pressure pulse curves are measured. These pressure oscillations or oscillation pressure signals no longer have any pulse contour. For example, an aortic valve closure (dicrotic notch) is no longer recognizable because the pulse curve contours are damped by the air used to transmit the tissue pressure signal and thus can no longer be detected by the sensor.

On the other hand, the non-invasive measurement of blood pressure values is characterized by an uncomplicated, fast, safe and cost-effective implementation and belongs to the daily medical routine, since in particular there is no risk for the patient in contrast to a direct, invasive blood pressure measurement.

In an invasive blood pressure measurement, an artery is punctured and a catheter is inserted. The catheter is connected to a pressure sensor so that the measured arterial blood pressure curve can be directly recorded and displayed on a monitor. The invasive blood pressure measurement is accurate compared to the non-invasive blood pressure measurement and is particularly suitable for continuous monitoring of critically ill patients and/or high-risk interventions. However, direct measurements exhibit particularly the risks of bleeding, thromboembolism, pseudoaneurysms, infections and nerve injuries, are expensive and time-consuming and are therefore mostly used to monitor and control blood pressure during surgeries and in intensive care.

Generally, a non-invasive, risk-free blood pressure measurement method is chosen over a risky, time-consuming and expensive invasive blood pressure measurement method, provided that the non-invasive method meets the requirements of accuracy, measurement frequency, reproducibility, and practicability. In addition, in order to reliably detect blood pressure values using an invasive blood pressure measurement method, it is necessary to continuously rinse the catheter for the invasive blood pressure measurement in order to remove continuously forming smallest blood clots at the catheter tip and to detect unaffected blood pressure curves using the free-communicating tube principle. In clinical practice, too little attention is often paid to the quality of the registered pressure curves due to a lack of available medical time, and even due to a lack of medical expertise, so that even in the invasive measurement method, blood pressure values which are quite different from one another are determined for one and the same original blood pressure curve. When used properly, the invasive blood pressure measurement method generally can provide more accurate measurement results than a non-invasive blood pressure measurement method. However, non-invasive blood pressure measurements are preferable for fast or ambulatory blood pressure monitoring.

In non-invasive blood pressure measurement methods using air-filled cuffs, in addition to tissue thickness and composition of the limb, the arterial stiffness of the patient and the quality of the coupling of the blood pressure measuring device to the tissue, the relative strength of the pulses of the blood pressure is also important. Thus, in situations with very low blood pressure in combination with a stiff artery and a thick tissue thickness, it is difficult to obtain correct results using a non-invasive blood pressure measurement method. Generally, very low blood pressure values come out too high and very high blood pressure levels come out too low, which results in a misleading of the physician and can endanger the patient.

Because of the considerable unreliability or lack of precision and comparability in known non-invasive blood pressure measurement methods, it is therefore necessary to provide an improvement in the determination of the blood pressure values for a non-invasive blood pressure measurement method in order to keep the advantages of a non-invasive blood pressure measurement over an invasive blood pressure measurement, in particular regarding costs, time consumption and absence of risks. Ideally, a non-invasive blood pressure measurement method should be so accurate and quickly repeatable or even continuous that it can replace invasive measurements with minimal compromises.

From U.S. Pat. No. 8,926,521 B2 it is for instance known to carry out an oscillation pressure measurement using a conventional pressure cuff and to calculate an upper and a lower envelope in order to estimate the systolic blood pressure value therefrom. Here, the maximum of the positive oscillation envelope curve is the mean blood pressure. Therefore, in conventional oscillometric blood pressure measurements using air-filled cuffs, it is necessary to determine the mean blood pressure first and then derive further values therefrom.

U.S. Pat. No. 5,606,977 A disclose an automated blood pressure monitoring, which uses a pneumatic cuff for performing a sphygmomanometric measurement on a patient. The mean and systolic blood pressures are determined.

It is an object of the invention to provide an improved method for non-invasively determining at least one blood pressure value. The object is solved by the features of the independent claims. Advantageous embodiments can be found in the dependent claims.

The invention proposes to detect a tissue pressure signal by means of a pressure cuff, wherein the tissue pressure signal comprises a sequence of tissue pressure pulse curves. According to the invention, it is provided to identify at least two tissue pressure pulse curves from the tissue pressure signal and to classify these tissue pressure pulse curves based on characteristic parameters.

The detection of the tissue pressure signal takes place over time or over the clamping pressure. For this purpose, the tissue pressure values supplied by a pressure sensor in the pressure cuff are recorded or stored together with the associated measuring times and/or clamping pressures. Having the pairs of values stored in this way, further processing of the tissue pressure signal is performed.

The stored pairs of values of tissue pressure and measuring time or clamping pressure can be pre-processed prior to further processing, for example, by disregarding pairs of values outside a trend. Various filter functions that are applied to the raw data can be used to create a database that is used for non-invasively determining blood pressure values according to the invention.

In addition to the detection of the pressure values over time or the clamping pressure, the identification may also include a graphic representation of the tissue pressure signal with the individual tissue pressure pulse curves. In order to identify one or more tissue pressure pulse curves from the tissue pressure signal, a recurring pattern is detected in the tissue pressure signal or in the pairs of values. For example, a lower and an upper tissue pressure envelope are determined in the tissue pressure signal by respectively connecting the adjacent tissue pressure systolic maxima or tissue pressure diastolic minima. For example, a tissue pressure pulse curve may be detected from a tissue pressure diastolic minimum to the following tissue pressure diastolic minimum. The successive tissue pressure diastolic minima in the tissue pressure signal represent respective end-diastolic points (time and pressure). In the following, a section of the tissue pressure signal, which extends from one end-diastolic point to the following end-diastolic point or whose associated pairs of values lie between these points, is considered as the tissue pressure pulse curve. If one considers the section from one end-diastolic point to the next end-diastolic point as the tissue pressure pulse curve, then the systole is in between, i.e., the tissue pressure pulse curve increases from the first end-diastolic point to the systole, where the tissue pressure signal values reach a local maximum each and then drop to the next end-diastolic point. The increasing and—to the aortic valve closure (characterized by an incisor, i.e., dicrotic notch)—decreasing portion of the tissue pressure pulse curve is referred to as the systolic section, and the portion, which decreases further after the dicrotic notch, is referred to as the diastolic section.

As described in detail later, a filter is applied to the detected tissue pressure signal, which either increases or decreases monotonously or gradually in the pressure region, or is kept constant for a certain time, to determine the clamping pressure. This clamping pressure is subtracted from the tissue pressure signal in order to filter out high frequency components from the tissue pressure signal for further processing, so that only the alternating component of the detected tissue pressure signal is used for the determination of the blood pressure values according to the invention. This provides a signal which fluctuates around a zero pressure point. A signal which has been processed in this manner enables a normalized or non-normalized further processing. In particular, comparable parameters can be determined therefrom for different tissue pressure pulse curves, which allow a reliable determination of the blood pressure values.

Based on the identified individual tissue pressure pulse curves, at least one amplitude parameter is determined for each identified tissue pressure pulse curve. The amplitude parameter represents a relationship between a tissue pressure diastolic minimum and a tissue pressure systolic maximum of a tissue pressure pulse curve. The amplitude parameter may comprise only a part between a tissue pressure diastolic minimum and a tissue pressure systolic maximum of a tissue pressure pulse curve.

Further, an area parameter is determined for each identified tissue pressure pulse curve, which is indicative of an area enclosed by the tissue pressure pulse curve. This can be either a partial area of the area enclosed by the tissue pressure pulse curve or the complete area enclosed by the tissue pressure pulse curve.

Based on the determined amplitude parameter and the area parameter, a pulsation power parameter is determined. The pulsation power parameter represents a characteristic value for a tissue pressure pulse curve.

In order to obtain the pulsation power parameter, the amplitude parameter and the area parameter are linked or set in relation to each other. Based on the pulsation power parameter, which results from the combination of the amplitude parameter and the area parameter, a parameter function is determined which indicates a relationship between the determined or derived pulsation power parameters of the respective identified tissue pressure pulse curves and the associated clamping pressures at the pressure cuff or the measuring times.

Based on the shape of the parameter function, characteristic values of the parameter function can be determined which are used for directly or indirectly determining blood pressure values according to the invention.

Using the method according to the invention, at least one of a systolic, mean and/or diastolic blood pressure value can be determined.

In order to detect the tissue pressure signal, the pressure cuff is provided with a clamping pressure over a predetermined pressure range from a low clamping pressure to a high clamping pressure or from a high clamping pressure to a low clamping pressure.

Further, it is possible to determine the tissue pressure signal only for a preset range or part of the predetermined pressure range from low to high clamping pressures or from high to low clamping pressures.

Preferably, the low clamping pressure is less than the diastolic blood pressure value and the high clamping pressure is higher than the systolic blood pressure value. Since the diastolic and the systolic blood pressure values are different for different patients, the low clamping pressure which is used as the initial pressure and the high clamping pressure which is used as the end pressure are set based on experience. In a preferred embodiment, the pressure range is quickly passed through using a first measuring method. This provides a preliminary systolic and/or diastolic blood pressure value in a fast manner. The associated diastolic or systolic blood pressure value can be determined based on the determined preliminary blood pressure value(s), so that the pressure range to be covered and the associated start and end values of the clamping pressure can be determined quickly. In a subsequent second measurement, the pressure range defined for the patient can then be passed through slowly in order to carry out the accurate measurements based on the detected tissue pressure signal. When passing through the pressure range from a high clamping pressure to a low clamping pressure, the end value and the start value are reversed.

In a preferred embodiment, the determined amplitude parameters of the tissue pressure pulse curves are multiplied by the associated area parameters in order to obtain the respective pulsation power parameter.

In a further advantageous embodiment, the pulsation power parameter can be determined for each tissue pressure pulse curve by assigning either the area parameter or the amplitude parameter or both with a preferred power. Here it is particularly advantageous to increase the amplitude parameter by a factor of three. However, powers in the range of −5 . . . 5 can also be selected.

In a further particular embodiment of the invention it is suggested that only a partial area enclosed by the tissue pressure pulse curve is used as the area parameter. Shape changes of the tissue pressure pulse curves show that, when the systolic pressure is passed through, the amplitude and the absolute area of the respective tissue pressure pulse curve decrease, and the shape of the upper ½ to 1/10 part of the pulse curve changes from round to pointed and that the tissue pressure systolic maximum can shift from late to early systolic. These changes affect the upper systolic part of the tissue pressure pulse curve. Therefore, systolic partial areas are defined, which are particularly sensitive to the passing of the systolic pressure.

In a preferred embodiment, a systolic upper partial area is determined based on a predetermined percentage amplitude value and a preferably horizontally extending line which intersects the (straightened, by the clamping pressure gradient corrected) tissue pressure pulse curve and forms a lower boundary of the partial area to be determined, wherein the partial area characterizing the systole then lies between the line and the tissue pressure pulse waveform.

In a further preferred embodiment, each pulsation power parameter is then assigned a measuring time or a clamping pressure, which are assigned to the respective tissue pressure pulse curve. This assignment is referred to in the following as the parameter function. This means that the parameter function maps the pulsation power parameter over the measuring time or the clamping pressure.

It is also advantageous to subject the determined parameter function to a smoothing method or to apply a curve fit to the determined pulsation power parameters in order to obtain a processable curve profile. For example, a Cauchy-Lorentz curve can be used.

In a further advantageous embodiment, it is possible to determine a first systolic blood pressure value using the determined parameter function. For this purpose, the maximum of the parameter function is determined. In addition, a first parameter function value is determined which follows the maximum of the parameter function in the case of a pressure curve from a low to a high clamping pressure and has a parameter function value which is reduced by a predetermined percentage with respect to the maximum. For the maximum parameter function value or the first parameter function value, the corresponding first measuring time or the corresponding first clamping pressure is determined.

When the pressure curve is passed from a high clamping pressure to a low clamping pressure, a parameter function value preceding the maximum is determined as a first parameter function value, which is also reduced by a predetermined proportion with respect to the maximum. Here, too, the associated first measuring time or the first clamping pressure is determined.

A corresponding first blood pressure value is determined from the tissue pressure signal by means of the first measuring time or the first clamping pressure recorded in this way. Here the upper envelope of the tissue pressure signal is preferably used to determine the first systolic blood pressure value at the first measuring time or the first clamping pressure from the tissue pressure signal.

The method according to the invention allows to determine a first mean blood pressure value by using the generated parameter function. Again, the maximum of the parameter function is determined. In the case of a pressure curve from a low to a high clamping pressure, a second parameter function value preceding the maximum is determined, which has a second parameter function value reduced by a predetermined proportion with respect to the maximum. Furthermore, the associated second measuring time or the associated second clamping pressure is determined. If the pressure goes from a high to a low clamping pressure, a second parameter function value following the maximum is determined, which has a parameter function value reduced by a predetermined proportion with respect to the maximum, and the associated second measuring time and/or the associated second clamping pressure is determined.

A corresponding second pressure value is determined or read from the tissue pressure signal on the basis of the second measuring time or the second clamping pressure. Here, the clamping pressure is preferably used in the tissue pressure signal to determine the corresponding first mean blood pressure value.

The method according to the invention allows to use the generated parameter function to determine a first diastolic blood pressure value. The maximum of the parameter function is determined. In the case of a pressure curve from a low to a high clamping pressure, a third parameter function value preceding the maximum is determined, which has a parameter function value reduced by a predetermined proportion with respect to the maximum, and the associated third measuring time or the associated third clamping pressure is determined.

If the pressure curve extends from a high to a low clamping pressure, a third parameter function value following the maximum is determined from the parameter function, which has a parameter function value reduced by a predetermined proportion with respect to the maximum, and the associated third measuring time or the associated third clamping pressure is determined.

Based on the determined third measuring time or the determined third clamping pressure, the corresponding pressure value is determined from the tissue pressure signal or a signal dependent thereon. The pressure value determined in this way corresponds to the first diastolic blood pressure value. Preferably, the first diastolic blood pressure value is determined from a lower envelope of the tissue pressure signal.

Based on the first systolic and first mean blood pressure values determined using the parameter function, it is possible to use an estimation formula to determine a second diastolic blood pressure value. For this purpose, the first mean blood pressure value and a difference between the first mean blood pressure value and the first systolic blood pressure value are multiplied by coefficients derived from invasive blood pressure measurements, their difference is formed and a correction constant derived from invasive blood pressure measurements is subtracted.

It is also possible to determine a second mean blood pressure value from the first systolic and first diastolic blood pressure values using another estimation formula obtained from invasive blood pressure measurements. For this purpose, the first diastolic blood pressure value and the difference between the first systolic blood pressure value and the first diastolic blood pressure value are multiplied by coefficients derived from invasive blood pressure measurements. A second correction constant derived from invasive blood pressure measurements is used to obtain a second mean blood pressure value.

In a preferred embodiment it is therefore possible to combine the first mean blood pressure value determined by the parameter function and the second mean blood pressure value determined by the estimation formula, preferably weighting and averaging them, in order to obtain a third mean blood pressure value. In this way, both a directly measured first mean blood pressure value and a second mean blood pressure value derived from the first diastolic or first systolic blood pressure value are determined, which are then linked together in such a way that a more resilient third mean blood pressure value can be obtained.

Similarly, the first diastolic blood pressure value determined from the parameter function and the second diastolic blood pressure value determined from the first mean or first systolic blood pressure value using the estimation formula, can be weighted to obtain an averaged third diastolic blood pressure value.

According to another aspect of the present invention, it is possible to obtain a second systolic blood pressure value from a tissue pressure signal using the identified tissue pressure pulse curves in the tissue pressure signal by determining a respective width parameter with respect to the tissue pressure pulse curve for a sequence of tissue pressure pulse curves. The width parameter characterizes a systole shape change of the tissue pressure pulse curves during the systole passage, particularly with respect to the maximum or the peak in the systole of the tissue pressure pulse curve. The systolic blood pressure value can be determined based on the change in systolic shape. To this end, the width parameter is determined based on an end-diastolic point of a previous tissue pressure pulse curve and a maximum of the current tissue pressure pulse curve. Alternatively, it is possible to determine the width parameter based on the maximum increase of the current tissue pressure pulse curve and the maximum of the current tissue pressure pulse curve. The width parameter is determined for multiple, preferably successive, tissue pressure pulse curves, wherein the associated measuring times or clamping pressures are determined. Further, it is determined at which measuring time or at which clamping pressure the width parameter shows a maximum change. The time at which the width parameter exhibits a maximum change over several tissue pressure pulse curves is the time at which the second systolic blood pressure value is determined from the tissue pressure signal or from a signal dependent thereon, preferably the clamping pressure. This means that at the measuring time or clamping pressure at which this width parameter changes the most, the second systolic blood pressure value can be derived from the tissue pressure signal, preferably from the clamping pressure of the tissue pressure signal.

In a preferred variant for the determination of a second systolic blood pressure value from a tissue pressure signal, the upper partial area is divided into an upper partial area located (in time) before a tissue pressure systolic maximum of the current tissue pressure pulse curve when the clamping pressure increases and an upper partial area located after (in time) a tissue pressure systolic maximum of the current tissue pressure pulse curve. For this purpose, the partial areas are formed as triangles. To form these two triangles, the tissue pressure pulse curve is delimited by a lower, preferably horizontal, straight line that intersects the tissue pressure pulse curve, wherein the tissue pressure pulse curve is straightened by filtering out the clamping pressure gradient. Furthermore, a common straight line is laid as a vertical line through the tissue pressure systolic maximum of the current tissue pressure pulse curve and a respective connecting straight line is laid between the intersection of the horizontal lower straight line with the tissue pressure pulse curve and the tissue pressure systolic maximum of the current tissue pressure-pulse curve. Thus, the two triangles for the determination of the partial areas are obtained.

This method can be carried out independently of the method described above regarding the parameter function. However, it can also be combined with the methods described above by determining the second systolic blood pressure value based on the time shift of the tissue pressure systolic maximum within the systole of the tissue pressure pulse curves in a sequence of successive tissue pressure pulse curves. From the two differently determined first and second systolic blood pressure values, a weighted average third systolic blood pressure value can be derived.

In order to determine the second systolic blood pressure value based on the temporal shift of the tissue pressure systolic maximum within the systole of the tissue pressure pulse curves, a moving mean value of the width parameter is determined over a predetermined number of tissue pressure pulse curves. Then a difference of the moving mean value of the width parameter and the individual width parameter for each tissue pressure pulse curve is determined. Based on these differences, a standard deviation function is generated for the individual tissue pressure pulse curves, and within this standard deviation function the center of the half-width of a developing bell form of the standard deviation function is determined from which the second systolic blood pressure value can be read at the center of the half-width.

When the area ratio of the two partial areas is used, a moving mean value of the area ratio of the two partial areas over a predetermined number of tissue pressure pulse curves is determined. Then a difference of the moving mean value of the area ratio of the two partial areas and the individual area ratio of the two partial areas for each tissue pressure pulse curve is determined. Based on these differences, a standard deviation function is generated for the individual tissue pressure pulse curves, and within this standard deviation function, the center of the half-width of a developing bell form of the standard deviation function is determined, from which the second systolic blood pressure value can be read at the center of the half-width.

In another aspect of the present invention, a method for non-invasively determining a fourth mean blood pressure value from a tissue pressure signal is provided. Multiple individual tissue pressure pulse curves are identified in the tissue pressure signal. The tissue pressure pulse curves together with limiting functions each enclose a surface. For successive tissue pressure pulse curves, a respective area is calculated up to the next tissue pressure pulse curve. The calculated area is divided into two partial areas, in particular into a partial area containing the systolic area and a diastolic partial area, wherein the partial area containing the systolic area lies below the tissue pressure pulse curve and the diastolic partial area lies above the tissue pressure diastolic minimum of the tissue pressure pulse curve. Based on a change in the area ratio of the systolic partial area and the diastolic partial area of successive tissue pressure pulse curves, the fourth mean blood pressure value can be determined from a corresponding tissue pressure signal, preferably the clamping pressure.

Preferably, it is possible to connect the method for determining the fourth mean blood pressure value, based on the area ratio, with the third mean blood pressure value. The third mean blood pressure value can thus be weighted and averaged with the fourth mean blood pressure value, and a fifth weighted mean blood pressure value can thus be determined.

In the method described above for identifying tissue pressure pulse curves, it is advantageous to subtract or filter the clamping pressure component from the tissue pressure signal in order to obtain the alternating component from the tissue pressure signal and thus transform the tissue pressure signal into a horizontally running signal curve. This allows a better comparability of the tissue pressure pulse curves and a better analysis of the individual parameters.

In the step of identifying tissue pressure pulse curves, at least two successive tissue pressure pulse curves are identified. To increase the reliability in regard to the blood pressure values, the number of identified and analyzed tissue pressure pulse curves can be increased.

Preferably, the pressure range is passed through during the measurement with a predetermined pressure change rate. The pressure range can preferably be determined during the measurement.

However, the pressure change rate can also be adjusted over time, so that, for example, measurements are made initially with a fast pressure change rate and subsequently with a slow pressure change rate.

The object is also solved by a measuring device for non-invasively determining blood pressure values, in which a tissue pressure signal is recorded by means of a pressure cuff on an individual, the measuring device comprising at least one control unit which is adapted to carry out the methods described above for determining the systolic, mean and/or diastolic blood pressure value.

A pressure cuff is preferably used to obtain the tissue pressure signal, wherein a pressure sensor is arranged in the pressure cuff and is hydraulically coupled to the tissue.

Further, the object is solved by a non-invasive blood pressure determination system comprising a pressure cuff having at least one pressure sensor configured to detect the tissue pressure signal on an individual, the system having a measuring device as described above for determining at least one blood pressure value from the detected tissue pressure signal. Preferably, the system may include a display unit for displaying the detected tissue pressure signal and the identified tissue pressure pulse curves.

In a further configuration, the measuring device can include a control unit which is configured to control a pressure transmitter in such a way that a pressure is dynamically built up and/or reduced at the pressure cuff over a pressure range determined during the measurement.

Particularly advantageous measurement results can be obtained if a shell wrapping cuff is used as the pressure cuff, which has an inner kink-resistant shell that hermetically encloses the extremity during the measurement and is hydraulically coupled to the tissue. In the shell wrapping cuff, hydraulically coupled transcutaneous tissue pressure pulse curves are detected with a pressure sensor located in/on the pressure cuff. In conventional pressure cuffs, no pressure sensor is arranged in the air-filled cuff. The pressure is transmitted via an air conduit to a measuring device where it is measured. Due to the transmission based on air, much of the information of the tissue pressure signal is damped and can therefore no longer be used for an evaluation. This means that for a high-quality measurement it is recommended to record the tissue pressure signal with the highest possible resolution.

It is advantageous to arrange a pressure sensor in the pressure cuff on the skin, without damping elements, e.g. air cushions, between them (hydraulic coupling). Protective films or, for reasons of compatibility, special substances between skin and sensor are possible, as they only minimally dampen the transmission of the tissue pressure pulse curve. Alternatively or additionally it is advantageous for the signal reception if the sensor is pressed by a solid and/or stiff element onto the skin. It is also advantageous if the tissue pressure pulse curve or the tissue pressure signal is detected as directly as possible hydraulically without using damping media for transmission.

With the method according to the invention or the combination of the different methods, non-invasive blood pressure values can also be determined for strongly hypotonic and hypertonic circulatory conditions, for intermittent arrhythmias, also on body parts with high tissue parts (e.g. body fat) which strongly dampen the signal transmission, and for contained or enclosed arteries with high stiffness.

In the following, the invention is explained in more detail using figures. Therein:

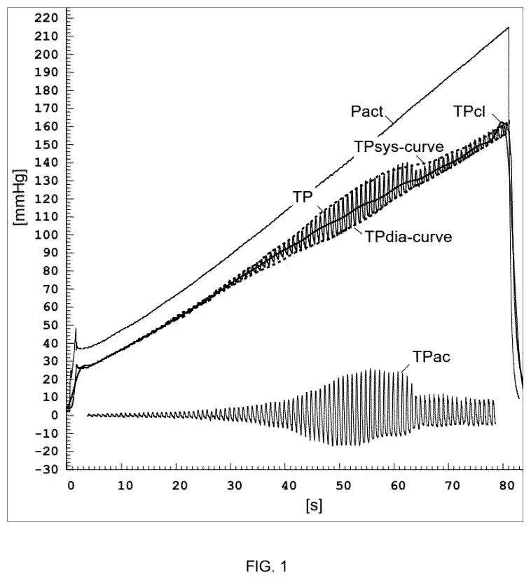

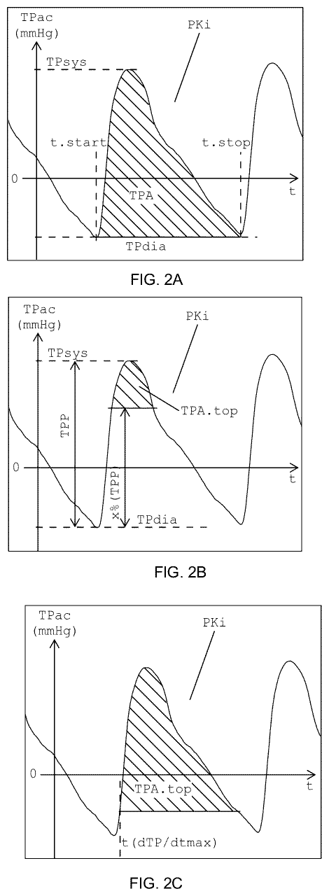

FIG. 1 shows a graphic representation of a tissue pressure signal, signals derived from it and the actuator pressure;

FIGS. 2A, 2B, 2C show each a tissue pressure pulse curve and parameters according to the first embodiment of the invention;

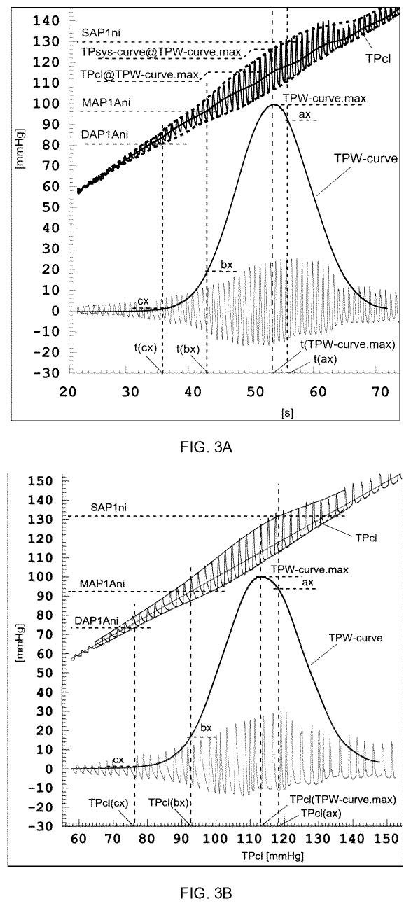

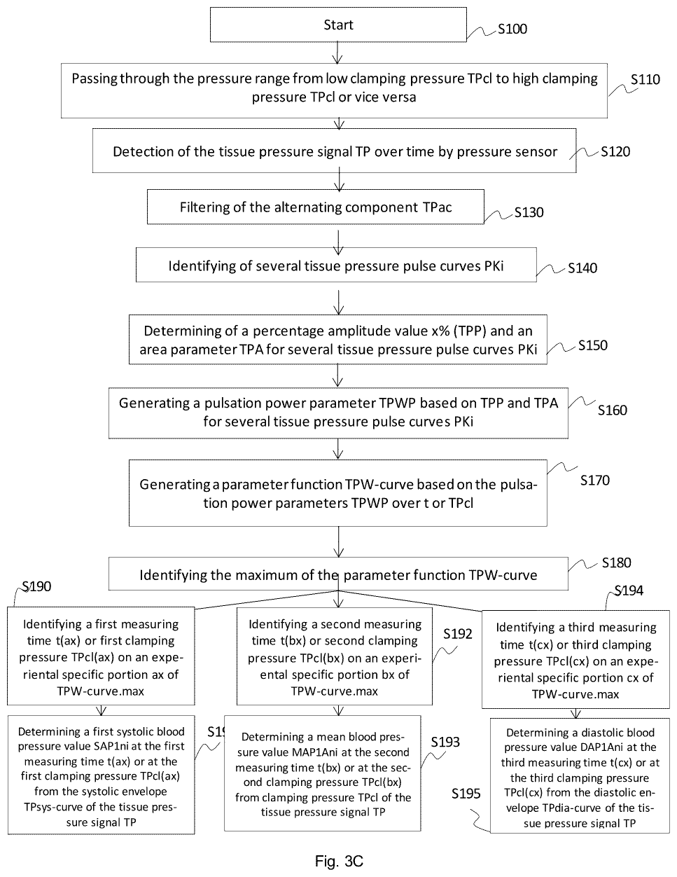

FIG. 3A shows a parameter curve derived from amplitude parameters and area parameters in accordance with FIGS. 2A-2C over time and blood pressure values derived therefrom;

FIG. 3B shows a parameter curve derived from amplitude parameters and area parameters according to FIGS. 2A-2C above clamping pressure and blood pressure values derived therefrom;

FIG. 3C shows a flow chart for carrying out the method according to the first embodiment;

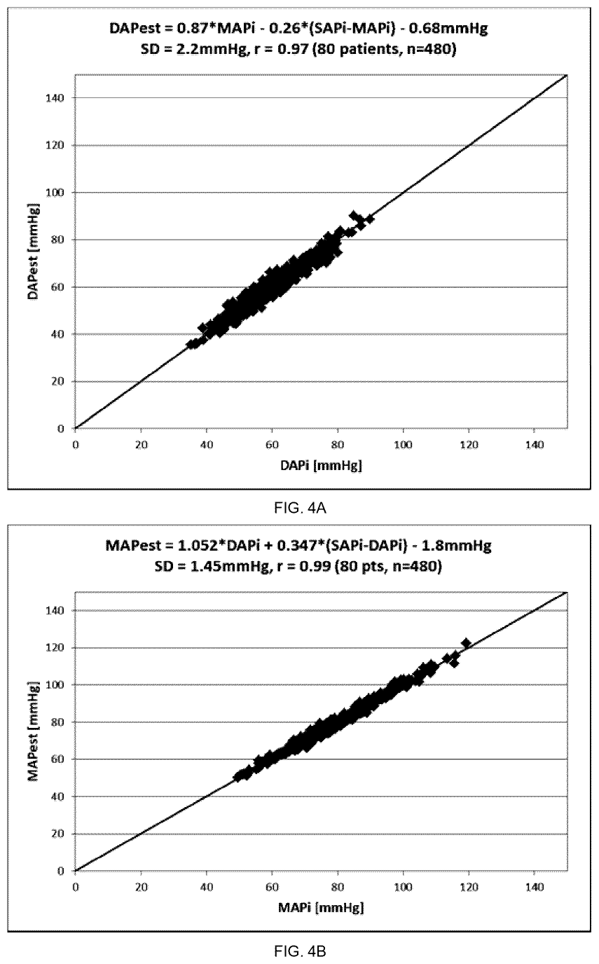

FIGS. 4A, 4B show each a correlation between blood pressure values determined by an estimation formula and invasively determined blood pressure values;

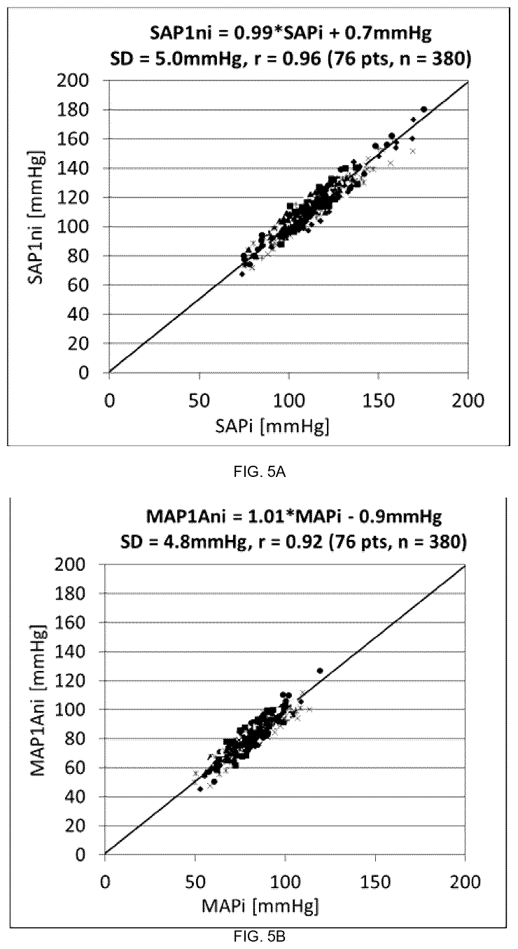

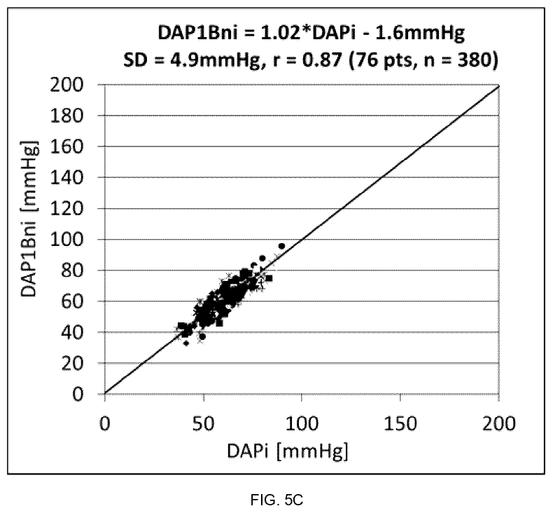

FIGS. 5A, 5B and 5C show each a correlation between non-invasively determined blood pressure values and invasively determined blood pressure values.

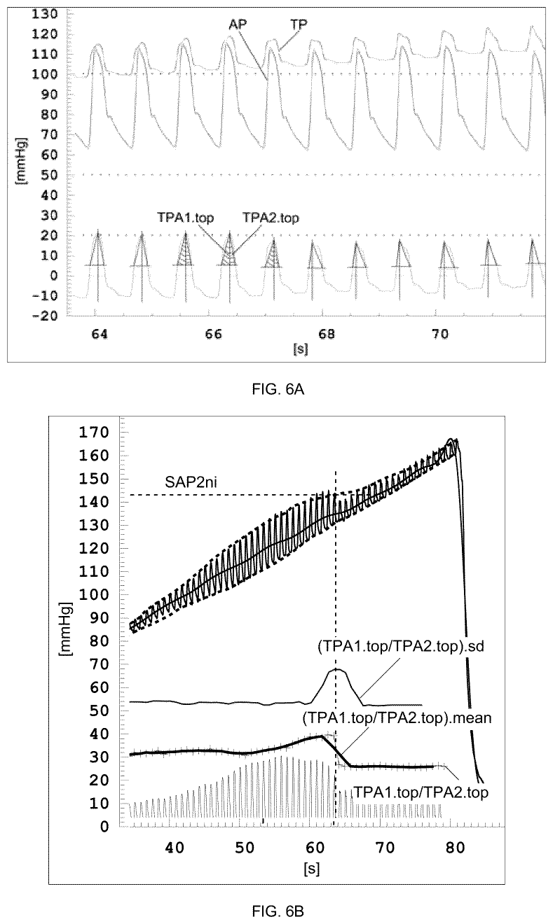

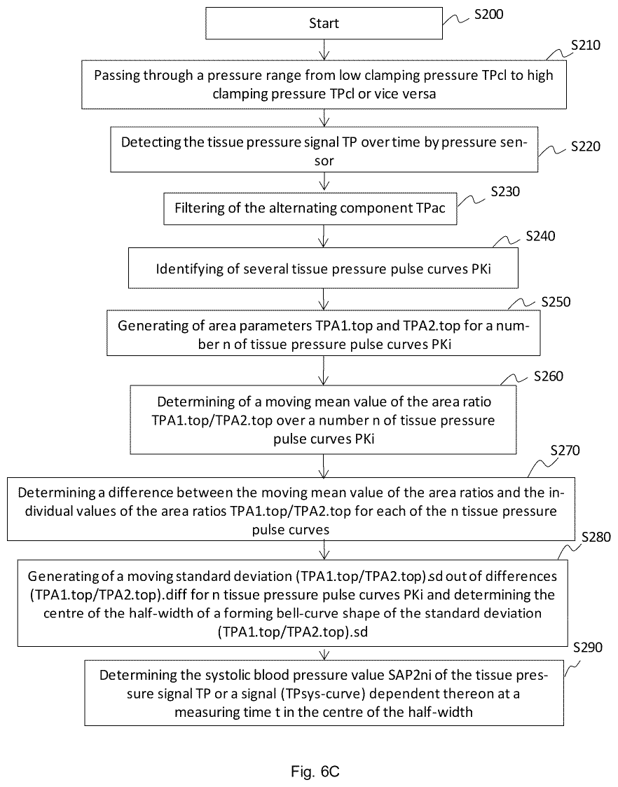

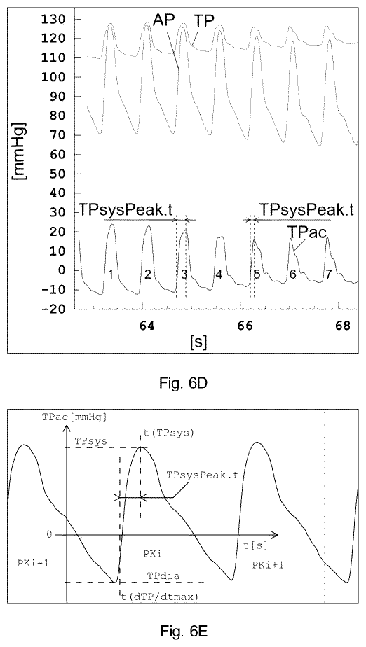

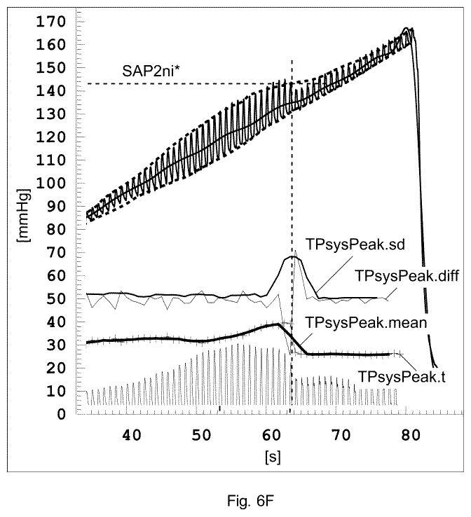

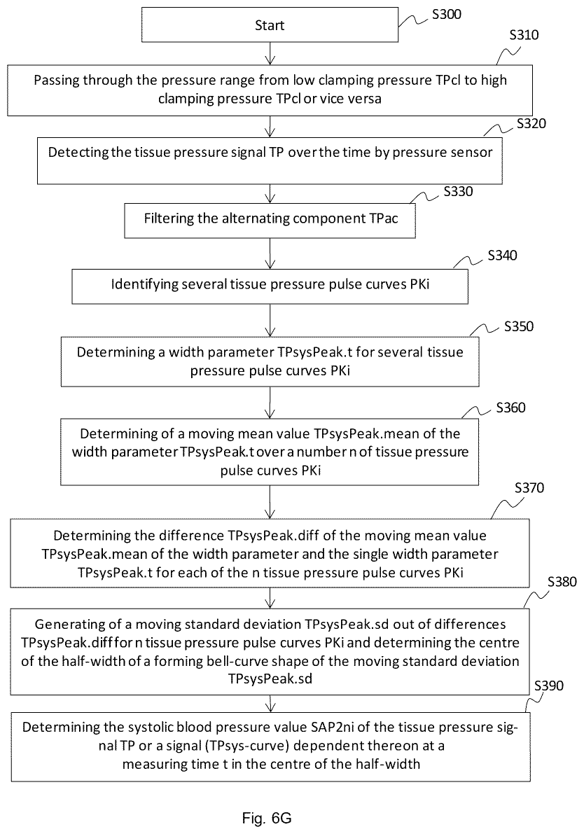

FIG. 6A shows tissue pressure pulse curves for determining the systole shape change of the tissue pressure pulse curves during the systole passage according to the second embodiment;

FIG. 6B shows a graphical representation for determining the systolic blood pressure value based on the change of the triangle area ratio according to the second embodiment;

FIG. 6C shows a flow chart for carrying out the method according to the second embodiment;

FIG. 6D shows tissue pressure pulse curves for determining the systole shape change of the tissue pressure pulse curves during the systole passage according to the third embodiment;

FIG. 6E shows an enlarged section of tissue pressure pulse curves to determine parameters for the third embodiment;

FIG. 6F shows a graphical representation for determining the systolic blood pressure value based on the change in the width parameter according to the third embodiment;

FIG. 6G shows a flow chart for performing the method according to the third embodiment;

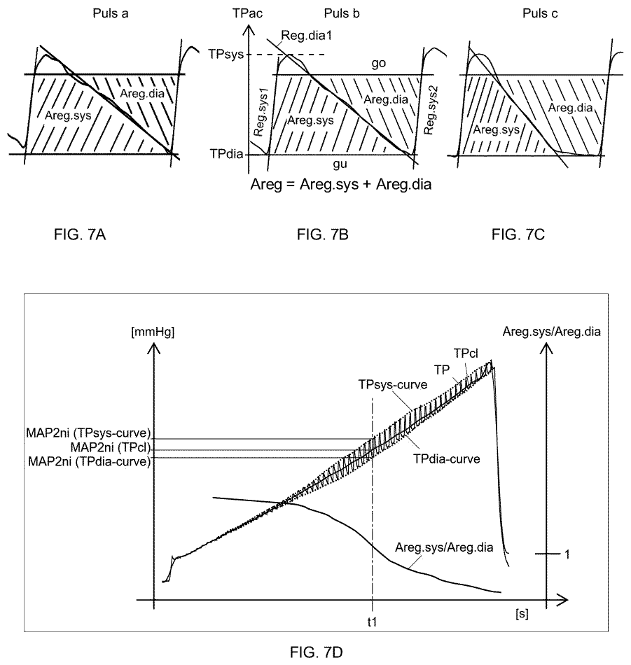

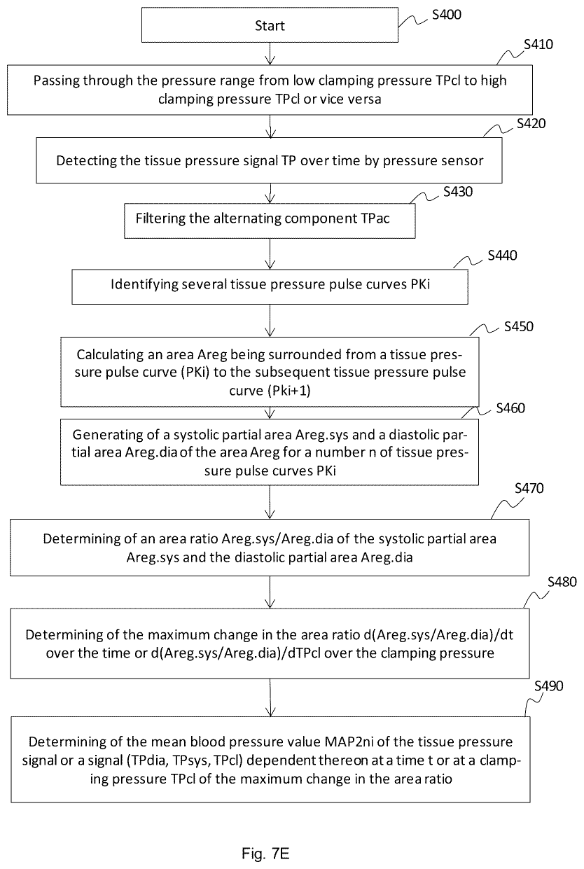

FIGS. 7A, 7B and 7C show tissue pressure pulse curves with different partial areas according to the fourth embodiment;

FIG. 7D shows a graphical representation for determining the mean blood pressure value based on the change of the partial area ratio according to the fourth embodiment;

FIG. 7E shows a flow chart for carrying out the method according to the fourth embodiment;

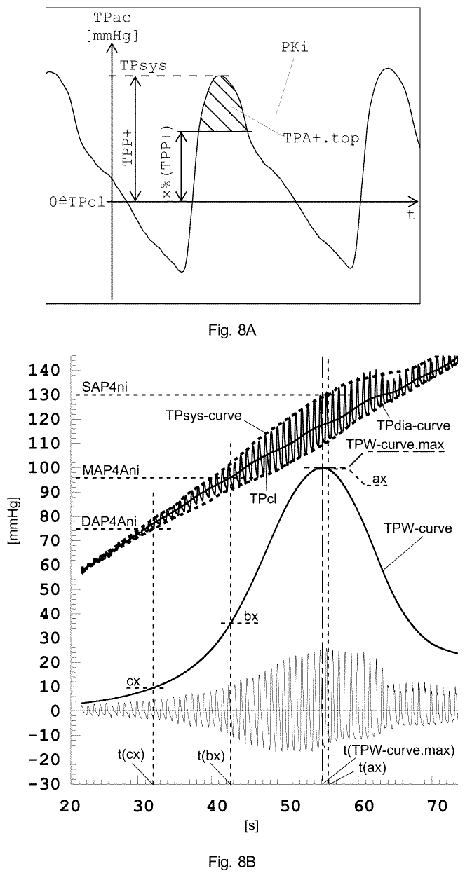

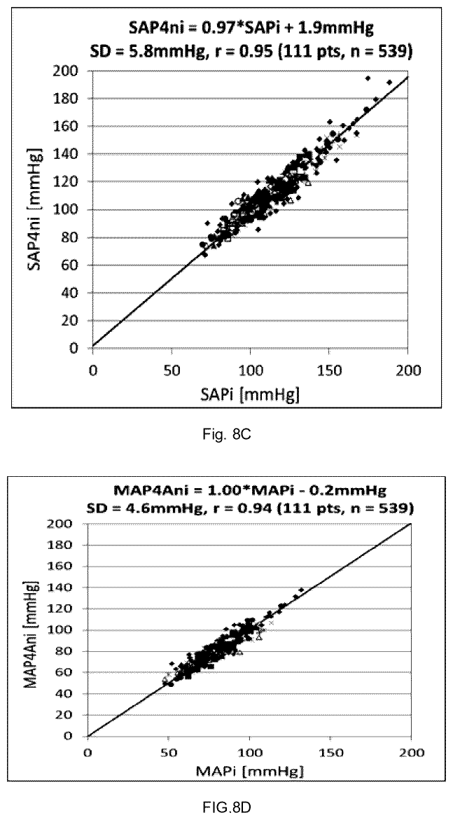

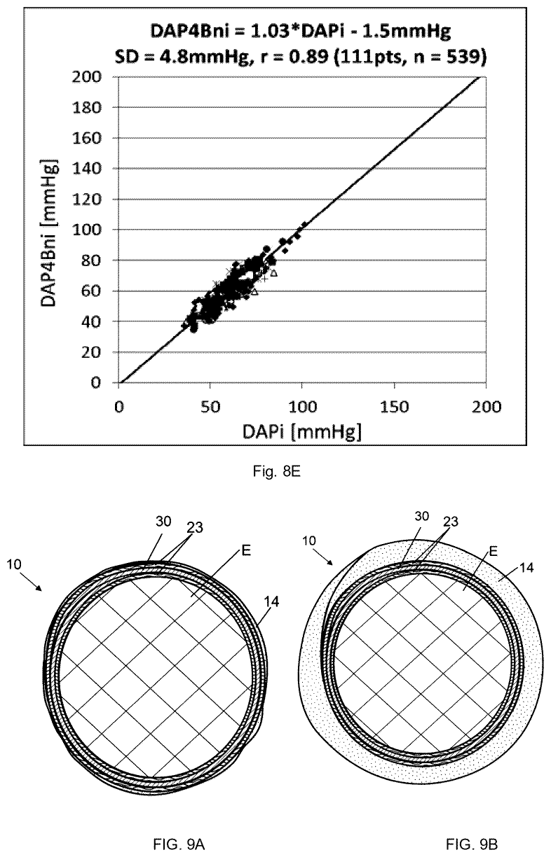

FIG. 8A shows a tissue pressure pulse curve and parameter according to an alternative embodiment of the invention based on the first embodiment;

FIG. 8B shows a parameter curve over time derived from amplitude parameters and area parameters according to FIG. 8A and blood pressure values derived therefrom;

FIGS. 8C, 8D, 8E show each a regression analysis between non-invasively determined blood pressure values and simultaneously invasively determined blood pressure values;

FIGS. 9A and 9B show sectional views of a shell pressure cuff;

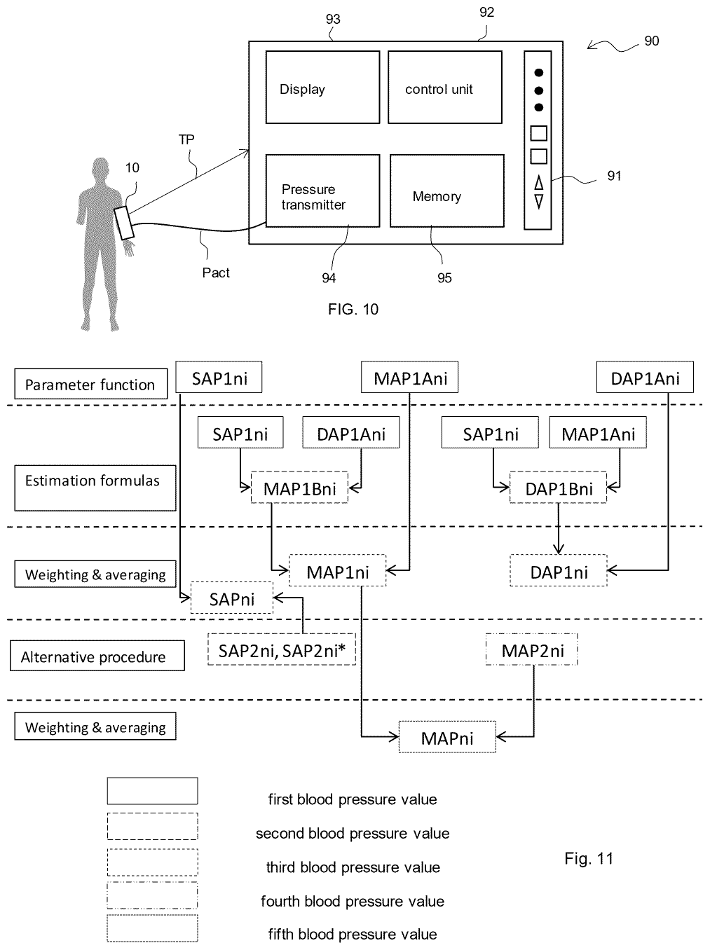

FIG. 10 shows the configuration of a system for non-invasive blood pressure determination;

FIG. 11 shows an overview of the combination of differently determined blood pressure values.

In the following, FIGS. 1, 2A-2C and 3A-3C are used to describe the first embodiment of a non-invasive determination of blood pressure values.

FIG. 1 shows the tissue pressure signal TP over time t. The actuator pressure Pact applied to the pressure cuff is shown in FIG. 1 and indicates the actuator pressure Pact delivered by a measuring device. It increases from a low value at 0 mmHg to 210 mmHg (S110).

The tissue pressure range typically covers a sufficiently large range from a low clamping pressure TPc1=0-20 mmHg to a high clamping pressure TPc1, wherein the high clamping pressure TPc1 is reliably above an empirical value or an online calculated systolic blood pressure value SAP1ni, SAP2ni or SAP2ni* and/or SAPni.

The non-invasively measured tissue pressure signal TP contains a sequence of high-resolution tissue pressure pulse curves PKi. The clamping pressure TPc1, which lies within the curve of the tissue pressure signal TP, is determined by low-pass filtering of the tissue pressure signal TP.

The pressure range can range from a low to a high clamping pressure TPc1 or vice versa (S110). The resulting tissue pressure signal TP, which is measured by the pressure sensor (S120), is shown in FIG. 1 and shows tissue pressure pulse curves PKi with changing amplitude. In addition to the tissue pressure signal TP, the clamping pressure TPc1 is shown, which increases analogously to the tissue pressure signal TP. FIG. 1 also shows the twofold alternating component TPac determined from the tissue pressure signal TP. With this alternating component TPac, which is obtained by filtering (S130), the tissue pressure pulse curves PKi can be better analyzed and a better comparability of the parameters determined from the tissue pressure pulse curves PKi is made possible. The alternating component TPac is preferably generated by subtracting the clamping pressure TPc1 from the tissue pressure signal TP.

As shown in FIG. 1, when the clamping pressure TPc1 increases, a usable tissue pressure signal TP is obtained from approx. 30 mmHg, which can be measured far beyond the systolic blood pressure value. Within this range the tissue pressure pulse curves PKi are identified (S140). Furthermore, FIG. 1 shows an upper envelope TPsys-curve of the tissue pressure signal TP obtained from the tissue pressure systolic maxima TPsys. A lower envelope TPdia-curve of the tissue pressure signal TP obtained from the tissue pressure diastolic minima TPdia is also shown.

FIG. 2A shows an identified tissue pressure pulse curve PKi in detail. The tissue pressure pulse curve PKi starts at an end-diastolic point, preferably at the local minimum of the tissue pressure pulse curve PKi, the tissue pressure diastolic minimum TPdia, and rises steeply to a maximum at the tissue pressure systolic maximum TPsys. The rising edge starting from the end-diastolic point to the tissue pressure systolic maximum TPsys and the falling edge of the tissue pressure signal TP from the tissue pressure systolic maximum TPsys to the next end-diastolic point includes the tissue pressure pulse curve PKi. This means that a tissue pressure pulse curve PKi extends from a start time t.start to a stop time t.stop. The pressure range that is crossed lies between the tissue pressure diastolic minimum TPdia and the tissue pressure systolic maximum TPsys. The area below the tissue pressure pulse curve PKi is referred to as the area parameter TPA and is delimited below the tissue pressure pulse curve by a straight line extending from the end-diastolic point from the start time t.start to the stop time t.stop. Preferably, the straight line is horizontal. In the case of a tissue pressure signal TP or an alternating component TPac thereof, the straight line for delimiting the area below the tissue pressure pulse curve PKi can also extend obliquely.

FIG. 2B shows a tissue pressure pulse curve PKi, analogous to FIG. 2A. A percentage amplitude valuex % (TPP) is shown, which ranges from the tissue pressure diastolic minimum TPdia to the percentage value of the tissue pressure systolic maximum TPsys. TPP indicates the whole amplitude from the tissue pressure diastolic minimum TPdia to the tissue pressure systolic maximum TPsys. The partial area TPA.top above this percentage amplitude value x % (TPP) can be used as area parameter TPA in the first embodiment of the invention. The percentage amplitude value x % (TPP) and the area parameter TPA are determined on the basis of the identified tissue pressure pulse curves PKi and the corresponding value pairs (S150).

FIG. 2C shows an alternative to the calculation of the partial area TPA.top below the tissue pressure pulse curve PKi. As an alternative to the method for determining the amplitude parameter TPP and area parameter TPA according to FIG. 2A or 2B, in this case the maximum increase dTP/dtmax or the time of the maximum increase t(dTP/dtmax) of the tissue pressure signal TP within a tissue pressure pulse curve PKi is determined. This point is used to determine the lower boundary of the partial area TPA.top. This means the range encompassed by the straight line at the point of maximum increase dTP/dtmax and the tissue pressure pulse curve PKi is used as area parameter TPA or as partial area TPA.top to calculate the first systolic blood pressure value SAP1ni, the first mean blood pressure value MAP1Ani and the first diastolic blood pressure value DAP1Ani.

A comparison of the determined blood pressure values based on the methods according to FIG. 2A, FIG. 2B or FIG. 2C shows that the use of the partial area TPA.top according to FIG. 2B or 2C generally results in more accurate blood pressure values, wherein the use of the method to determine the amplitude parameter TPP and the partial area TPA.top according to FIG. 2B generally results in the most reliable blood pressure values.

In order to obtain the pressure values of the tissue pressure signal TP in mmHg over time, the tissue pressure signal TP is recorded by a pressure sensor and stored and processed in the measuring device with high resolution, wherein a tissue pressure signal value is being detected at each measuring time t or clamping pressure TPc1 in accordance with the set resolution, and wherein these values are being stored together in a memory of the measuring device as value pairs.

To further describe the method according to the first embodiment, it is referred to FIG. 3A. FIG. 3A shows a parameter function TPW-curve which is determined from the product of the amplitude parameter TPP and the area parameter TPA or the partial area TPA.top above the percentage amplitude value x % (TPP) for each tissue pressure pulse curve PKi (S170).

A range of 50-90% of the TPP, preferably 75% of the TPP, has proven to be particularly advantageous for the percentage amplitude value x % (TPP) of the first embodiment.

Based on the amplitude and area parameters TPP and TPA or the partial area TPA.top determined for each identified tissue pressure pulse curve PKi, a pulsation power parameter TPWP can be calculated (S160) by connecting the amplitude parameter TPP or a proportion x % (TPP) thereof with the area parameter TPA or the partial area TPA.top.

To this end, the amplitude parameter TPP or a fraction x % (TPP) thereof and the area parameter TPA or the partial area TPA.top are used for each identified tissue pressure pulse curve PKi as factors, which are weighted with one exponent each to form a pulsation power parameter TPWP. The pulsation power parameter TPWP is provided in the simplest form as a product of the amplitude parameter TPP and the area parameter TPA, preferably based on the formula:

$$TPWP = {TPA}^{exp 1} \cdot {TPP}^{exp 2}$$

$$where exp 1 \neq 0 , exp 2 \neq 0$$

Alternatively, the pulsation power parameter TPWP can also be calculated according to the formula:

$$TPWP = TPA . {top}^{exp 1} \cdot TP {P}^{exp 2} \cdot {( dTP / dtmax )}^{exp 3}$$

$$where exp 1 \neq 0 , exp 2 \neq 0 , exp 3 \neq 0$$

The parameter function TPW-curve shown in FIGS. 3A and 3B is derived from the values determined for the pulsation power parameter TPWP (S 170). For this purpose, each determined pulsation power parameter TPWP is assigned to the corresponding measuring time t or the corresponding value derived from the tissue pressure signal TP that belongs to the identified tissue pressure pulse curve PKi. This means that each value of a pulsation power parameter TPWP is assigned a time or tissue pressure signal value of the associated tissue pressure pulse curve PKi, preferably the time of the tissue pressure systolic maximum t(TPsys) is assigned as the time, alternatively the clamping pressure TPc1, the tissue pressure systolic maximum TPsys or the tissue pressure diastolic minimum TPdia is assigned. A smoothed parameter function TPW-curve, as shown in FIGS. 3A and 3B, is generated by a low-pass filtering of the parameter function, e.g. by means of a multi-stage and continuous averaging over the clamping pressure TPc1 or by means of a multi-stage and continuous averaging over e.g. 6 to 10 seconds.

The parameter function generated in this way or its value pairs can be analyzed and certain function values of the parameter function can be determined which are used to determine the blood pressure values according to the invention.

The parameter function TPW-curve has a maximum parameter function value TPW-curve.max, which is identified (S180). Based on experience, a first measuring time t(ax) is determined which belongs to a first parameter function value ax which includes a predetermined portion of the maximum parameter function value TPW-curve.max (S190). Based on the first measuring time t(ax), a first systolic blood pressure value SAP1ni is determined (S191) on the basis of the upper envelope TPsys-curve of the tissue pressure signal TP, wherein the pressure value belonging to the first measuring time t(ax) in the tissue pressure signal TP is determined or read off. In FIG. 3A, the first measuring time t(ax) is 56 s and is on increasing pressure curve behind the maximum, which is at 53.5 s. The first measuring time t(ax) is 56 s and, with an increasing pressure curve, behind the maximum, which is 53.5 s. Based on the first measuring time t(ax) of 56 s, the corresponding tissue pressure signal TP is determined to determine the first systolic blood pressure value SAP1ni, which in the present case is at TPsys-curve=130 mmHg.

Alternatively, as it is shown in FIG. 3A, a pressure value TPc1@TPW-curve.max is obtained at the ordinate in FIG. 3A at a time t(TPW-curve. max) from the assigned clamping pressure TPc1 of the tissue pressure signal TP when the maximum parameter function value t(TPW-curve.max) occurs. Based on experience a specific factor TPc1% is applied to TPc1@TPW-curve.max to determine an alternative first systolic blood pressure value SAP1ni*.

In another alternative, as it is shown in FIG. 3A, a pressure value TPsys-curve@TPW-curve.max is obtained at the ordinate in FIG. 3A corresponding to the upper envelope of the tissue pressure signal TP (TPsys-curve is defined in FIG. 1) at the time of the occurrence of the maximum parameter function value t(TPW-curve.max). Based on experience a specific factor TPsys-curve % is applied to TPsys-curve@TPW-curve.max to determine another alternative first systolic blood pressure value SAP1ni**.

The parameter function can also be used to determine a first mean blood pressure value MAP1Ani, wherein, with an increasing pressure curve, a second parameter function value bx of the parameter function TPW-curve and the associated second measuring time t(bx) are determined (S192). The associated second measuring time t(bx) is 43 s in FIG. 3A. The corresponding first mean blood pressure value MAP1Ani is determined based on the clamping pressure TPc1 (S193) and in this case is approx. 96 mmHg.

Analogous to the first systolic blood pressure value SAP1ni and the first mean blood pressure value MAP1Ani, the diastolic blood pressure value DAP1Ani can be determined based on the parameter function TPW-curve by determining a third parameter function value cx reduced by a predetermined proportion and the associated third measuring time t(cx) (S194), which is 36 s in the present case. Based on the third measuring time t(cx), the corresponding pressure value of approx. 80 mmHg is determined or read off (S195) of the tissue pressure signal TP, and in particular of the lower tissue pressure envelope TPdia-curve.

In FIG. 3B the tissue pressure signal TP is shown above the clamping pressure TPc1 and in the lower area of FIG. 3B a twofold alternating component TPac determined therefrom. Analogous to the method according to FIG. 3A, based on the identified tissue pressure pulse curves PKi and the amplitude parameter TPP and area parameter TPA determined therefrom, the pulsation power parameter TPWP is determined for each tissue pressure pulse curve PKi first, and from the pulsation power parameters TPWP the parameter curve TPW-curve over the clamping pressure TPc1 is determined as shown in FIG. 3B.

In contrast to FIG. 3A, the parameter function TPW-curve, which represents the pulsation power parameter TPWP from the combination of the area parameter TPA and the amplitude parameter TPP, is not represented over time t in FIG. 3B, but as a function of the clamping pressure TPc1. The clamping pressure TPc1 is less susceptible to drift or disturbances in the lower envelope or baseline (TPdia-curve) caused, for example, by motion artifacts, muscle tremors or tension in awake patients or individuals.

Similar to the parameter function according to FIG. 3A, the parameter function TPW-curve in FIG. 3B has a maximum which is identified for the determination of the blood pressure values (S180) and in particular the corresponding clamping pressure TPc1(TPW-curve.max). Depending on this maximum, a first, second and/or third parameter function value ax, bx, cx is determined (S190, S192, S194), which lies before or after the maximum of the parameter curve, depending on the pressure curve of the clamping pressure TPc1. The associated clamping pressures TPc1(ax), TPc1(bx) and TPc1(cx) are determined for each of these parameter function values ax, bx, cx, which each have a predetermined proportion of the maximum parameter function value TPW-curve.max, wherein the predetermined proportion is determined empirically or experimentally. A corresponding blood pressure value (S191, S193, S195) is determined in the tissue pressure signal TP or a signal dependent thereon (TPdia-curve, TPsys-curve, TPc1) by means of these clamping pressure values for the three parameter function values.

Thus, a first systolic blood pressure value SAP1ni can be determined using the first clamping pressure TPc1(ax) to determine the corresponding blood pressure value using the upper envelope TPsys-curve of the tissue pressure signal TP. In the example shown in FIG. 3B, at a TPc1(ax) clamp pressure of 118 mmHg, a systolic blood pressure value of 132 mmHg is determined based on the upper envelope TPsys curve as the first systolic blood pressure value SAP1ni.

Analogous to the method according to FIG. 3A, the first mean blood pressure value MAP1Ani can be determined for the second clamping pressure TPc1(bx) at 92 mmHg at the clamping pressure TPc1 of the tissue pressure signal TP and is 92 mmHg in this example.

The diastolic blood pressure value DAP1Ani is determined with the aid of the third parameter function value cx, the associated third clamping pressure TPc1(cx) of which is 76 mmHg, wherein the corresponding diastolic blood pressure value DAP1Ani is determined using the lower envelope TPdia-curve of the tissue pressure signal TP, so that a diastolic blood pressure value DAP1Ani of approximately 73 mmHg is obtained.

In order to obtain the values for the first to third parameter function values ax, bx and cx, a calibration data set is created from the same number of simultaneous invasive and non-invasive blood pressure measurements on a sufficient number of individuals in different cardiovascular states.

An overview of the method according to the first embodiment is shown in FIG. 3C.

FIGS. 4A and 4B show estimated diastolic and mean blood pressure values DAPest and MAPest, which were determined based on invasively determined blood pressure values using an estimation formula. In FIG. 4A, the estimated values are shown as a set of points around the regression line for the estimated diastolic blood pressure value DAPest, which were determined by means of the estimation formula from the invasively determined mean blood pressure MAPi and the invasively determined systolic blood pressure SAPi, as a function of the invasively determined diastolic blood pressure DAPi.

Thus, FIG. 4A shows a correlation between the estimated diastolic blood pressure values DAPest based on invasively determined systolic and mean blood pressure values SAPi and MAPi and the corresponding invasively measured diastolic blood pressure values DAPi based on a data set of 480 measurements on 80 patients. To determine the estimated diastolic blood pressure value DAPest, the following equation, determined by regression analysis of invasive blood pressure values, was applied:

$$DAPest = 0.87 \cdot MAPi - 0.26 \cdot ( SAPi - MAPi ) - 0.68 mmHg .$$

The coefficients (0.87 and 0.26) and the correction constant (0.68 mmHg) were determined empirically by determining the systolic and mean blood pressure values SAPi and MAPi of a number of patients by statistically evaluating as large a data set of invasive clinical blood pressure measurements as possible.

Thus, it was found that the diastolic blood pressure value DAPest can be reliably derived or estimated from the systolic and mean blood pressure values. The representation according to FIG. 4A thus shows that the estimated values for the diastolic blood pressure value DAPest deviate insignificantly from the invasively determined comparative values for the diastolic blood pressure value DAPi, wherein the standard deviation SD of the differences DAPest−DAPi is 2.2 mmHg and the correlation coefficient r is 0.97.

FIG. 4B shows, analogous to FIG. 4A, the determination of an estimated value for the mean blood pressure value MAPest, based on invasively determined diastolic and systolic blood pressure values DAPi and SAPi. The equation used here is:

$$MAPest = 1.052 \cdot DAPi + 0.347 \cdot ( SAPi - DAPi ) - 1.8 mmHg .$$

As can be seen in FIG. 4B, the estimate is even more accurate than the one shown in FIG. 4A, since the correlation coefficient r is 0.99. The points of the estimated values for the mean blood pressure value MAPest is even closer to the regression line than in FIG. 4A. The standard deviation SD of the differences MAPest−MAPi is 1.45 mmHg.

FIGS. 5A, 5B and 5C show a comparison of a simultaneous invasive arterial measurement and non-invasive tissue pressure measurement as structural regression diagrams for the parameters systolic, mean and diastolic blood pressure values.

FIG. 5A, shows the blood pressure values SAP1ni determined using the first method of FIG. 3C based on the parameter function vs. the simultaneously determined invasively determined blood pressure values SAPi. It is clearly visible that the various measuring points for the systolic non-invasively determined values differ insignificantly from the invasively determined values.

FIG. 5B also shows the first mean blood pressure values MAP1Ani determined using the first method of FIG. 3C based on the parameter function vs. the corresponding simultaneously determined invasive mean blood pressure values MAPi. Here, too, it is clearly evident that the various measuring points for the mean non-invasively determined values deviate insignificantly from the invasively determined values.

FIG. 5C shows values for the estimated diastolic blood pressure value DAP1Bni vs. the corresponding simultaneously invasively determined diastolic blood pressure values DAPi. The estimated diastolic blood pressure values DAP1Bni are determined from the first systolic blood pressure values SAP1ni determined according to FIG. 3C and the first mean blood pressure values MAP1Ani, based on the parameter function.

The following estimation formula is used:

$$DAP 1 Bni = k 1 \cdot MAP 1 Ani - k 2 \cdot ( SAP 1 ni - MAP 1 Ani ) - k 3 mmHg ,$$

$$wherein k 1 = ( 0.6 … 1 , 1 ) , k 2 = ( 0 \text{.15} … 0.4 ) and k 3 = ( - 5 … 5 ) .$$

FIG. 5C clearly shows that the various measuring points for the estimated diastolic blood pressure values DAP1Bni deviate insignificantly from the invasively determined diastolic values.

Similar to the determination of the estimated diastolic blood pressure value DAP1Bni, an estimated second mean blood pressure value MAP1Bni can be determined. The following estimation formula is used for this purpose:

$$MAP 1 Bni = k 4 \cdot DAP 1 Ani + k 5 \cdot ( SAP 1 ni - DAP 1 Ani ) - k 6 mmHg .$$

$$wherein k 4 = ( 0.8 … 1.3 ) , k 5 = ( 0.25 … 0.5 ) , k 6 = ( - 5 … 5 ) .$$

results of statistical evaluations of SAP1ni, MAP1ni, DAP1ni

| | | | structural regression & | | | Bland- | |
|---|---|---|---|---|---|---|---|
| | | | correlation | | | Altman | |
| | | n/patient | increase | intercept | r | mean | SD |
| SAP1ni vs SAPi | ax = 92.4% | 380/76 | 0.99 | 0.7 | 0.96 | −0.31 | 5.0 |
| MAP1Ani vs MAPi | bx = 19.4% | 380/76 | 1.01 | −0.9 | 0.92 | −0.30 | 4.8 |
| DAP1Bni vs DAPi | | 380/76 | 1.02 | −1.6 | 0.87 | −0.27 | 4.9 |

ni = non-invasive; i = invasive; n = 5 ni/i measurements/patient in high-risk surgery; intercept = y-axis section; r = correlation coefficient; ax = % TPWmax after TPWmax (with increasing clamping pressure); bx = % TPWmax before TPWmax (with increasing clamping pressure); mean = mean value of the differences between non-invasive and invasive; SD = standard deviation of the differences of non-invasive and invasive.

FIGS. 6A, 6B and 6C show a preferred method for the determination of the second systolic blood pressure value SAP2ni, which is essentially based on an accurate recognition of the shape change of systoles of the tissue pressure pulse curves PKi during systolic passage. With increasing clamping pressure TPc1, the systole passage indicates the closure of the arteries enclosed by the cuff, and with decreasing clamping pressure TPc1 the systole passage indicates the opening of the arteries enclosed by the cuff.

The upper part of FIG. 6A shows an invasively measured arterial blood pressure signal AP and a non-invasively measured tissue pressure signal TP. In the lower part of FIG. 6A, the non-invasively measured tissue pressure pulse curves PKi are filtered, i.e. the increasing clamping pressure TPc1 has already been removed, so that only the alternating component TPac of the tissue pressure signal TP is shown. It is clearly visible that, over time, the peak of the tissue pressure systolic maximum TPsys shifts from the right (late systolic) to the left (early systolic). The tissue pressure systolic maximum TPsys of the tissue pressure pulse curves PKi at 64s is almost centered or inclined to the right. In the right part of FIG. 6A the tissue pressure systolic maximum TPsys of the tissue pressure pulse curves PKi is strongly inclined to the left.

Observations of the shape changes of the tissue pressure pulse curves PKi in FIG. 6A show that, across the systolic pressure (closure of the arteries enclosed by the cuff), the amplitude and the absolute area decrease and, in particular, the shape of the upper pulse pressure part of the pulse curve changes from round to pointed, and in a few cases to double-peak/pointed always with a dominant peak. It can also be seen that in most cases investigated, the tissue pressure systolic maximum TPsys shifts from middle to late to early systolic due augmentation when the systolic pressure is passed. In the second most frequent case of the patients examined, it showed that the tissue pressure systolic maximum TPsys shifts from medium to late systolic during arterial closure to far late systolic and remains there in the suprasystolic clamping pressure range. In rare cases, the tissue pressure systolic maximum TPsys shifts from middle to late systolic during arterial closure and jumps back and forth between early and late systolic and then remains in the suprasystolic clamping pressure range approximately in the middle of the tissue pressure pulse curve.

In all these cases it is possible to reliably determine a second systolic blood pressure value SAP2ni using the method described in the second embodiment of FIGS. 6A to 6C.

For this purpose, an area ratio TPA1.top/TPA2.top is obtained, which is obtained from partial areas TPA1.top and TPA2.top (S250). First, a partial area TPA.top is obtained below the tissue pressure pulse curve PKi, wherein the tissue pressure pulse curve PKi is intersected at approx. 50% of the maximum amplitude measurement TPP by a preferably horizontal straight line. Then a vertical line is placed at the tissue pressure systolic maximum TPsys of the current tissue pressure pulse curve PKi. Furthermore, connecting straight lines are arranged to the left and right, which run from the tissue pressure systolic maximum TPsys to the point of intersection of the current tissue pressure pulse curve PKi with the lower straight line. In this way, two triangles are formed having the triangular partial areas TPA1.top and TPA2.top. The two partial areas TPA1.top and TPA2.top can be calculated so that an area ratio TPA1.top/TPA2.top can be derived therefrom. The change of the area ratio TPA1.top/TPA2.top is used to determine the second systolic blood pressure value SAP2ni.

Based on the area ratios TPA1.top/TPA2.top obtained for the sequence of multiple tissue pressure pulse curves PKi, a moving mean value of the area ratio TPA1.top/TPA2.top.mean is determined (S260), which is shown in FIG. 6B. Preferably this moving mean value of the area ratio TPA1.top/TPA2.top.mean is determined over five tissue pressure pulse curves PKi. Subsequently, for each tissue pressure pulse curve PKi the difference TPA1.top/TPA2.top.diffbetween the moving mean value of the area ratio TPA1.top/TPA2.top.mean and the individual values of the area ratio TPA1.top/TPA2.top is determined (S270). Since the difference TPA1.top/TPA2.top.diff scatters more strongly during the systolic passage than immediately after and before it, the scatter can be used to accurately determine the systolic blood pressure value. To detect the change in scattering, a moving standard deviation TPA1.top/TPA2.top.sd of the differences TPA1.top/TPA2.top.diff is typically determined over three to seven, preferably over five, differences TPA1.top/TPA2.top.diff (S280) as shown in FIG. 6B. The moving standard deviation TPA1.top/TPA2.top.sd is plotted over time or over the clamping pressure TPc1 of the associated tissue pressure pulse curves PKi, preferably over the time t. Alternatively, the clamping pressure TPc1 or the upper envelope TPsys-curve or lower envelope TPdia-curve of the tissue pressure signal TP can be used. As shown in FIG. 6B, the moving standard deviation TPA1.top/TPA2. top.sd indicates that a bell-shaped increase occurs during the systolic passage. Furthermore, the moving standard deviation TPA1.top/TPA2.top.sd is characterized by the fact that it is essentially flat before and after the bell-shaped elevation. Thus, for the reliable determination of the second systolic blood pressure value SAP2ni using the method of the second embodiment, it is possible to determine the beginning and the end of the bell-shaped elevation. The start and end points of the half-width are preferably determined, wherein, at the point in the middle between the start and end point, the time or the clamping pressure for the second systolic blood pressure value SAP2ni can be determined, or at the maximum of the moving standard deviation TPA1.top/TPA2.top.sd, based on which the second systolic blood pressure value SAP2ni is then determined based on the upper tissue pressure envelope TPsys-curve of the tissue pressure signal TP (S290).

FIGS. 6D to 6G illustrate a method for determining a different or alternative second systolic blood pressure value SAP2ni*, based on a third embodiment of the invention. Analogous to the method according to the first embodiment, a tissue pressure signal TP is recorded at an increasing or decreasing clamping pressure TPc1 (S310), wherein individual tissue pressure pulse curves PKi are recorded. From this tissue pressure signal TP, the alternating component TPac is filtered out or extracted (S330) by means of filtering, and which is used for further processing. Based on the alternating component TPac, individual tissue pressure pulse curves PKi are identified (S340). In this aspect, the method according to the third embodiment corresponds to the method according to embodiment 1.

In the method according to the third embodiment, a second systolic blood pressure value SAP2ni* is determined, wherein the temporal shift of the tissue pressure systolic maximum TPsys is determined.

FIG. 6D shows a non-invasively measured tissue pressure signal TP compared to an arterially measured pressure signal AP over time. It can be clearly seen that the signal stroke of the non-invasively measured tissue pressure signal TP is lower than that of the arterially measured pressure signal AP. In the upper part of FIG. 6D, the waveform of the tissue pressure signal TP indicates that the tissue pressure systolic maximum TPsys within a tissue pressure pulse curve PKi moves from a temporally late systole to a temporally early systole when passing the systolic blood pressure.

The lower part of FIG. 6D shows a magnification of the non-invasive tissue pressure signal TP, wherein only the alternating component TPac is considered. Several tissue pressure pulse curves 1 to 7 are shown. Here, too, it is clearly visible that the tissue pressure systolic maximum TPsys in the tissue pressure signal TP shifts from a late systole to an early systole. Based on this finding, the second systolic blood pressure value SAP2ni* can be determined, wherein the time is detected at which the systole or tissue pressure systolic maximum TPsys changes from a late systole to an early systole.

In order to detect the crossing of the systolic blood pressure value by the change of the tissue pressure systolic maximum TPsys, a width parameter TPsysPeak.t of several tissue pressure pulse curves PKi is determined according to FIG. 6D (S350). The width parameter TPsysPeak.t changes in the course of a sequence of tissue pressure pulse curves PKi, as shown in FIG. 6D. For example, in a tissue pressure pulse curve 3, the width parameter TPsysPeak.t is much greater than in tissue pressure pulse curve 5, where the tissue pressure systolic maximum TPsys has already changed from a late systole to an early systole.

In order to accurately determine the width parameter TPsysPeak.t, according to FIG. 6E, the time of the maximum increase t(dPT/dtmax) in the systolic flank of the tissue pressure pulse curve is determined after the identification of a tissue pressure pulse curve PKi using the tissue pressure diastolic minima TPdia, which each represent a minimum of a tissue pressure pulse curve. The time of the maximum increase t(dPT/dtmax) of the tissue pressure pulse curve t(dPT/dtmax) characterizes the start parameter for calculating the width parameter TPsysPeak.t. The end point of the width parameter TPsysPeak.t is defined by the tissue pressure systolic maximum TPsys.

Based on the width parameters TPsysPeak.t determined for the sequence of several tissue pressure pulse curves PKi, a moving mean value TPsysPeak.mean (S360) is determined, which is shown in FIG. 6F. Preferably, the moving mean value TPsysPeak.mean is determined over five tissue pressure pulse curves PKi. Subsequently, for each tissue pressure pulse curve PKi the difference TPsysPeak.diff of the moving mean value TPsysPeak.mean and the individual values TPsysPeak.t for each pulse curve is determined (S370). Since the difference TPsysPeak.diff scatters more strongly at the systole passage than immediately after and before it, the scatter can be used to accurately determine the systolic blood pressure value.

To determine the change in scattering, a moving standard deviation TPsysPeak.sd of the differences TPsysPeak.diff is determined typically over three to seven, preferably over five, differences TPsysPeak.diff (S380), as shown in FIG. 6F. The moving standard deviation TPsysPeak.sd is mapped over time or over the clamping pressure TPc1 of the associated tissue pressure pulse curves PKi, preferably using the times of the tissue pressure systholic maxima t(TPsys) as time. Alternatively, the clamping pressure TPc1 or the upper envelope TPsys-curve or lower envelope TPdia-curve of the tissue pressure signal TP can be used.

As shown in FIG. 6F, the moving standard deviation TPsysPeak.sd shows that a bell-shaped increase occurs during the systole passage. Furthermore, it is characteristic for the moving standard deviation TPsysPeak.sd to be essentially flat before and after the bell-shaped elevation. Thus, for the reliable determination of the second systolic blood pressure value SAP2ni* using the method described in the third embodiment, the beginning and the end of the bell-shaped elevation can be determined. Preferably, the start and end points of the half-width are determined, wherein at the point in the middle between the start and end point the time for the second systolic blood pressure value SAP2ni* can be determined, or in the maximum of the moving standard deviation TPsysPeak.sd, by which the second systolic blood pressure value SAP2ni* can then be determined based on the upper envelope TPsys-curve of the tissue pressure signal TP (S390).

As an alternative to the upper envelope TPsys-curve of the tissue pressure signal TP, the clamp pressure TPc1 or the lower envelope TPdia-curve of the tissue pressure signal TP can be used to determine the second systolic blood pressure value SAP2ni* based on the time or clamp pressure in the middle between the start and end point or at the maximum of the bell-shaped increase in the values of the moving standard deviation TPsysPeak.sd. In FIG. 6G, the sequence of the method according to the third embodiment is shown again as a flow diagram.

According to a fourth embodiment of the present invention, a method for determining the fourth mean blood pressure value MAP2ni is determined on the basis of FIGS. 7A to 7D, and which based on a change in the area ratio of multiple tissue pressure pulse curves PKi, wherein in particular a relative area ratio of the systolic area of the tissue pressure pulse curve Areg.sys to the diastolic area of the tissue pressure pulse curve Areg.dia is determined.

As shown in FIG. 7A, a tissue pressure pulse curve PKi has a systolic partial area Areg.sys and a diastolic partial area Areg.dia. The systolic partial area Areg.sys is generally the area which lies below a tissue pressure pulse curve PKi between two end diastolic points. The diastolic partial area Areg.dia is the area above the tissue pressure pulse curves PKi and PKi+1 between their tissue pressure systolic maxima TPsys.

In the method according to the invention, the areas Areg.sys and Areg.dia are determined by determining an upper and a lower straight line go and gu respectively, wherein the upper straight line go is positioned at a predetermined percentage amplitude value and preferably extends horizontally. Preferably, a percentage amplitude value of 75% of the amplitude parameter TPP is used to delimit the systolic and diastolic area of the tissue pressure pulse curves Areg.sys and Areg.dia upwards. To this end, it is necessary that the upper straight line go for limiting the systolic and diastolic areas Areg.sys and Areg.dia for all tissue pressure pulse curves PKi, for each of which an area ratio Areg.sys/Areg.dia is determined, is provided at the same percentage amplitude value.

The lower straight line gu is positioned on the end diastolic point of the following tissue pressure pulse curve PKi+1.

Preferably, the upper straight line go lies between the tissue pressure diastolic minimum TPdia and the tissue pressure systolic maximum TPsys of the respective tissue pressure pulse curve PKi, and preferably at a height of the tissue pressure diastolic minimum TPdia+75% TPP.

It is also necessary for the lower straight line gu to be at the same tissue pressure diastolic minimum TPdia of the respective following tissue pressure pulse curve PKi+1 for all tissue pressure pulse curves PKi which are considered.

The upper and lower straight lines go, gu define the total area Areg. In a subsequent step, the area Areg, which is composed of the systolic and diastolic partial areas Areg.sys. and Areg.dia, is divided by the regression line Reg.dial, which is approximated to the falling flank of the tissue pressure pulse curve PKi.

Furthermore, a first regression line Reg.sys1, based on the considered tissue pressure pulse curve PKi, is determined, which limits the increasing portion of the tissue pressure pulse curve PKi.

Preferably, the first regression line Reg.sys1 is provided from values in the range of 20 to 80% of the amplitude parameter TPP. Furthermore, a second regression line Reg.sys2 is determined which simulates the increasing portion of the following tissue pressure pulse curve PKi+1, wherein it is too provided from values in the range of 20 to 80% of the amplitude parameter TPP. By using the upper and lower straight lines go, gu of the first and second regression lines Reg.sys1 and Reg.sys2 and the falling straight line Reg.dial1, which divides the area Areg of the tissue pressure pulse curves PKi and PKi+1 in the systolic partial area Areg.sys and the diastolic partial area Areg.dia, it is possible to calculate the area of the systolic partial area Areg.sys and the diastolic partial area Areg.dia and to compare these partial areas. Thus, an area ratio Areg.sys/Areg.dia can be determined for each tissue pressure pulse curve PKi. The area ratio Areg.sys/Areg.dia changes the most at the point where the clamping pressure TPc1 crosses the mean blood pressure MAP.

FIGS. 7A, 7B and 7C show that the diastolic partial area Areg.dia increases from FIG. 7A to FIG. 7C with respect to the total area Areg during inflation of the pressure cuff. This means that the area ratio Areg.sys/Areg.dia decreases during inflation of the pressure cuff with increasing area of the diastolic partial area Areg.dia. It was found out that, prior to the passage of the clamping pressure TPc1 through the mean blood pressure in FIG. 7A, an area ratio Areg.sys/Areg.dia occurs, which is >1. In the vicinity of the passage of the clamping pressure through the mean blood pressure (FIG. 7B) the area ratio Areg.sys/Areg.dia between the systolic partial area Areg.sys and the diastolic partial area Areg.dia is almost one. After the passage of the clamping pressure through the mean blood pressure MAP (FIG. 7C), the area ratio Areg.sys/Areg.dia is <1.

If the pressure range is covered from a high to a low clamping pressure TPc1, i.e. if the pressure cuff is deflated, the area ratio Areg.sys/Areg.dia increases accordingly.

As shown in FIG. 7D, the area ratio Areg.sys/Areg.dia can be used to determine when the area ratio Areg.sys/Areg.dia is close to one or shows its largest change. At this time ti, the fourth mean blood pressure value MAP2ni can be determined using the tissue pressure signal TP or a signal dependent therefrom (TPsys-curve TPdia-curve, TPc1). Preferably, the fourth mean blood pressure value MAP2ni is determined or read from at clamping pressure TPc1 of the tissue pressure signal TP. In FIG. 7E, the sequence of the method according to the fourth embodiment is shown again as a flow diagram.

FIGS. 8A-8E describe a fifth embodiment in which systolic, mean and/or diastolic blood pressure values, SAPni, MAPni, DAPni, are determined non-invasively based on other pulsation power parameters TPWP. Analogous to the first embodiment, the pulsation power parameter TPWP is obtained based on an amplitude parameter and an area parameter.

As shown in FIG. 8A, a tissue pressure pulse curve PKi is used to determine the amplitude parameter, which is referred to below as the positive amplitude parameter TPP+. In comparison to FIG. 2A and the first embodiment where the amplitude parameter TPP was used, only the positive portion TPP+ of the amplitude TPP between diastolic and systolic blood pressure is used in this embodiment. This is thus the fraction between the clamping pressure TPc1=0 and the tissue pressure systolic maximum TPsys. The positive amplitude parameter TPP+ is the positive fraction of TPP in a tissue pressure pulse curve PKi, in a tissue pressure signal TPac straightened by TPc1 and corrected for slope. TPP is the full amplitude from the tissue pressure diastolic minimum TPdia to the tissue pressure systolic maximum TPsys (shown in FIG. 2B).

In addition, an area parameter is determined from the tissue pressure curve PKi, analogous to the first embodiment. In the fifth embodiment, however, a positive area parameter TPA+.top is determined from the tissue pressure curve PKi, unlike in the first embodiment.

The positive area parameter TPA+.top indicates the area of a tissue pressure pulse curve PKi, which is delimited in the upper part by TPsys and in the lower part by a preferably horizontal straight line, which lies in the range of TPac≥0, e.g. by a horizontal line at x % of TPP+. The value x % (TPP+) can be in the range from 0 to 90% TPP+.

The alternative pulsation power parameter according to the fifth embodiment is obtained based on the area parameter TPA+.top and the amplitude parameter TPP+, namely

$$TPWP = TPA + . {top}^{e x p 1} \cdot TPP {+}^{exp 2} ,$$

wherein exp1≠0, exp2≠0 which are experimentally determined.

The pulsation power parameter TPWP is determined for a plurality of tissue pressure pulse curves PKi, resulting in the parameter function TPW-curve shown in FIG. 8B. The area parameter TPA+.top and the amplitude parameter TPP+ are determined for each tissue pressure pulse curve PKi as well as the corresponding time, whereby the bell-shaped form of the parameter function TPW-curve is obtained.

Then, based on the previously determined parameter function values ax, bx and cx, the corresponding sixth systolic, mean and/or diastolic blood pressure values SAP4ni, MAP4Ani and DAP4Ani are obtained as alternatives for SAP1ni, MAP1Ani and DAP1Ani of the first embodiment.

Alternatively, SAP4ni* and SAP4ni** are determined analogously to SAP1ni* and SPA1ni** by applying a specific factor TPc1+% to TPc1@TPW-curve.max or applying a specific factor TPsys-curve+% to TPsys-curve@TPW-curve.max.

The parameter function value ax is close to the maximum of TPW-curve. At a time t(ax), a sixth systolic blood pressure value SAP4ni is determined from TPsys-curve, which corresponds to the pressure value at the intersection of t(ax) with TPsys-curve.

The parameter function TPW-curve can also be used to determine a fourth mean blood pressure value MAP4Ani, wherein, at an increasing pressure curve, a second parameter function value bx of the parameter function TPW-curve and the associated second measuring time t(bx) are determined.

The associated second measuring time t(bx) is 42.5 s in FIG. 8B. The corresponding sixth blood pressure value MAP4Ani is determined on the basis of the clamping pressure TPc1 and in the present case is approx. 96 mmHg.

Analogous to the sixth systolic blood pressure value SAP4ni and the sixth mean blood pressure value MAP4Ani, the sixth diastolic blood pressure value DAP4Ani can also be determined on the basis of the parameter function TPW curve by determining a third parameter function value cx reduced by a predetermined proportion and the associated third measuring time t(cx), which here is 32 s. Based on the third measuring time t(cx), the corresponding pressure value of approx. 75 mmHg is determined or read from the lower tissue pressure envelope TPdia-curve.

Analogous to the first embodiment and the second diastolic blood pressure value DAP1Bni determined therein, in the fifth embodiment a seventh diastolic blood pressure value DAP4Bni is calculated as follows based on the sixth mean blood pressure value MAP4Ani and the sixth systolic blood pressure value SAP4ni, which is hereinafter also referred to as the estimated or derived seventh diastolic blood pressure value.

$$DAP 4 Bni = k 1 \cdot MAP 4 Ani - k 2 \cdot ( SAP 4 ni - MAP 4 Ani ) - k 3 mmHg ,$$

$$wherein k 1 = ( 0.6 … 1.1 ) , k 2 = ( 0 , 15 … 0.4 ) and k 3 = ( - 5 … 5 ) .$$

Furthermore, similar to the first embodiment and the second mean blood pressure value MAP1Bni determined therein, in the fifth embodiment a seventh mean blood pressure value MAP4Bni is calculated as follows based on the sixth diastolic blood pressure value DAP4Ani and the sixth systolic blood pressure value SAP4ni.

$$MAP 1 Bni = k 4 \cdot DAP 4 Ani + k 5 \cdot ( SAP 4 ni - DAP 4 Ani ) - k 6 mmHg ,$$

$$wherein k 4 = ( 0.8 … 1.3 ) , k 5 = ( 0.25 … 0.5 ) , k 6 = ( - 5 … 5 ) .$$

FIG. 8B shows an example of a parameter function TPW-curve based on

$$TPWP = TPA + . {top}^{0.5} \cdot TPP {+}^{1.}$$

The area parameter TPA+.top is limited by a horizontal line at 50% of TPP+, wherein the corresponding sixth blood pressure values SAP4ni, MAP4Ani and DAP4Ani are as follows:
ax=99.8% from TPWmax to TPWmax,
bx=36.5% of TPWmax before TPWmax,
cx=9.5% of TPWmax before TPWmax.

FIGS. 8C, 8D, 8E show the results of the regression analyses of the sixth blood pressure values SAP4ni, MAP4Ani and the derived seventh diastolic blood pressure value DAP4Bni of 539 measurements on 111 patients determined according to the fifth embodiment vs. their corresponding simultaneously determined invasive reference values SAPi, MAPi and DAPi.

It can be seen that SAP4ni in FIG. 8C, MAP4Ani in FIG. 8D and the derived seventh diastolic blood pressure value DAP4Bni can achieve the same high accuracies of the blood pressure values as SAP1ni, MAP1Ani and DAP1Bni according to the first embodiment.

Specifically, FIG. 8C shows a regression analysis which, based on the method according to the fifth embodiment, shows sixth systolic blood pressure values SAP4ni determined based on the parameter function of FIG. 8B vs. simultaneously invasively determined systolic blood pressure values SAPi. FIG. 8D shows a regression analysis which, based on the method according to the fifth embodiment, shows sixth mean blood pressure values MAP4Ani determined based on the parameter function of FIG. 8B vs. simultaneously invasively determined systolic blood pressure values SAPi.

In FIG. 8E a regression analysis is shown in which the seventh diastolic blood pressure value DAP4Bni derived or estimated from the sixth mean blood pressure value MAP4Ani and the sixth systolic blood pressure value SAP4ni was used vs. the diastolic blood pressure values DAPi determined simultaneously invasively, wherein SAP4ni and MAP4Ani were determined based on the parameter function in accordance with FIG. 8B.

FIGS. 9A and 9B show a shell pressure cuff 10 which is particularly suitable for the methods described above for recording the tissue pressure pulse curves PKi. In FIG. 9A the shell pressure cuff 10, which is also referred to as a shell wrapping cuff, is shown in a pressureless state, whereby in FIG. 9B the shell pressure cuff is shown under pressure.

The shell pressure cuff 10 has a kink resistant or buckling resistant shell 30 which is arranged inside the shell pressure cuff 10. The shell 30 is arranged under or between the pressure generating means and a body part E. The pressure generating means are formed by a fluid-tight shell 14. When an air pressure is supplied to the pressure generating means, the kink resistant shell 30 is pressed against the body part E. A textile layer can also be arranged between body part E and the kink resistant shell 30. The pressure sensor (not shown) for recording the tissue pressure signal TP is arranged on the inner circumference of the shell 30 below the textile layer 23, so that the textile layer isolates the sensor from the body part E. This ensures that the pressure sensor rests directly on the body part, couples hydraulically to it and there are no other damping materials in between.

The pressure sensor (not shown) is connected to an electrical pressure receiver by means of a fluid line, which can receive a pressure change transmitted via the fluid within the fluid line (not shown) and convert it into an electrical signal, the tissue pressure signal TP.

FIG. 10 shows a measuring device 90 of the invention which is connected to the shell pressure cuff 10. The measuring device 90 comprises a control unit 92, a memory 95, a display 93 and a pressure transmitter 94. Furthermore, a display and operating device 91 is provided, which is configured for controlling the measuring device and comprises setting regulators, on and off buttons and display elements.

The display 93 shows the tissue pressure signal TP detected by the measuring device 90. In addition, an enlarged view of the identified tissue pressure pulse curves PKi can be shown on the display 93. The control unit 92 records the tissue pressure signal TP over time or over the clamping pressure TPc1 and stores the corresponding value pairs in a memory 95.

Based on the blood pressure value to be recorded, one of the methods described according to the invention is carried out by, based on the detected tissue pressure signal TP and the corresponding times or clamping pressures TPc1, determining corresponding tissue pressure pulse curves PKi and corresponding parameters based thereon.

The control unit 92 also controls the pressure transmitter 94, which applies an actuator pressure Pact to the pressure cuff, preferably a shell blood pressure cuff 10. As described above, the tissue pressure signal TP is detected by the pressure cuff 10 by means of a pressure sensor (not shown), wherein the pressure signal is transmitted via a fluid to an electrical pressure receiver (not shown) and an electrical pressure signal is provided to the measuring device 90 in order to display and evaluate the tissue pressure signal TP.

FIG. 11 shows how the blood pressure values determined with the various methods are connected to each other in order to obtain stable or resilient blood pressure values. As described above, a first systolic blood pressure value SAP1ni and a first mean blood pressure value MAP1Ani and a first diastolic blood pressure value DAP1Ani can be determined by means of the parameter function according to the first embodiment.

These determined values can be linked by means of empirically determined estimation formulas as described above, from which both a second mean blood pressure value MAP1Bni and a second diastolic blood pressure value DAP1Bni can be determined. This means that the second mean blood pressure value MAP1Bni is determined using the estimation formula from the first systolic blood pressure value SAP1ni according to the parameter function and the first diastolic blood pressure value DAP1Ani according to the parameter function. The second diastolic blood pressure value DAP1Bni is determined using an estimation formula from the first systolic blood pressure value SAP1ni and the first mean blood pressure value MAP1Ani.

From the second mean blood pressure value MAP1Bni determined by the estimation formula and the first mean blood pressure value MAP1Ani which is based on the parameter function, a third mean blood pressure value MAP1Ani can be determined by weighting and averaging.

Analogously, a third diastolic blood pressure value DAP1ni is obtained by weighting and averaging of the second diastolic blood pressure value DAP1Bni determined by the estimation formula and the first diastolic blood pressure value DAP1Ani determined by the parameter function.

By means of a weighting, and according to certain quality criteria, the third mean blood pressure value MAP1ni and/or the third diastolic blood pressure value DAP1ni can be improved, taking into account the second mean blood pressure value MAP1Bni and/or the second diastolic blood pressure value DAP1Bni with regard to accuracy. The weighting can preferably be done in such a way that, in proportion to the percentage size of the amount of the difference of the first mean blood pressure value MAP1Ani and the second mean blood pressure value MAP1Bni, the portion of the first mean blood pressure value MAP1Ani is weighted higher. Weighting of the portions of DAP1Ani and DAP1Bni can be done accordingly.

The first systolic blood pressure value SAP1ni obtained by the parameter function is linked to the second systolic blood pressure value SAP2ni or SAP2ni* determined by systolic shift according to the second or third embodiment. Here, weighting and averaging are performed to obtain a resilient third systolic blood pressure value SAPni.

Similarly, the third weighted and averaged mean blood pressure value MAP1ni described above is linked by weighting and averaging to the fourth mean blood pressure value MAP2ni, which was calculated using the partial area calculation according to the third embodimentle. From this, the fifth mean blood pressure value MAPni is obtained.

By means of weighting, and according to certain quality criteria, the third systolic blood pressure value SAPni and/or the fifth mean blood pressure value MAPni can be improved taking into account the second systolic blood pressure value SAP2ni or SAP2ni* and/or the fourth mean blood pressure value MAP2ni with regard to accuracy. The weighting can preferably be done in such a way that, in proportion to the percentage size of the amount of the difference of the first systolic blood pressure value SAP1ni and the second systolic blood pressure value SAP2ni or SAP2ni*, the portion of the first systolic blood pressure value SAP1ni is weighted higher. Weighting of the portions of MAP1ni and MAP2ni can be done accordingly.

To compensate for differences in the sizes and physical properties of pressure cuffs, which can also be specifically referred to as blood pressure cuffs, a specific correction or calibration can preferably be performed. Particularly in the case of a hydraulic adjustment of different shell wrapping cuff designs, e.g. with regard to size, strength or thickness of the shells, a correction with specific coefficients can be carried out.

As an example, the combination of SAP1ni and MAP1Ani is used to demonstrate this according to the following dependencies:

$$SAP 1 ni . corr = coeff 1 \cdot SAP 1 ni + const 1$$

$$MAP 1 Ani . corr = coeff 2 \cdot MAP 1 Ani + const 2$$

The correction coefficients and constants coeff1, const1, coeff2, const2 can be obtained by calibration in comparison with reference values, in particular invasive reference values, preferably with coeff1,2: 0.7 . . . 1.5 and const1,2: −20 . . . 20.

FIGS. 5A and 5B show regression diagrams that show the comparison of values SAP1ni.corr, MAP1Ani.corr (referred to in the diagram as SAP1ni and MAP1Ani) determined according to the method described above with simultaneously invasively measured values SAPi and MAPi from a selected, broad set of clinical measurement data, each with the same number of simultaneous invasive and non-invasive measurements. The data are based on 380 measurements on 76 patients. The formulas in the diagrams represent the equations of the regression lines. r indicates the correlation coefficient of the respective regression and SD indicates the standard deviation differences SAP1ni−SAPi or MAP1Ani−MAPi.

In the following, a control of the clamping pressure increase and/or the clamping pressure decrease at the blood pressure cuff is described.

In one embodiment, the clamping pressure TPc1 on the blood pressure cuff can be quickly built up. As already described above, the clamping pressure TPc1 can be either increasing or decreasing after rapid inflation.

The acquisition of signals (tissue pressure signal TP) can thus take place with increasing and/or decreasing clamping pressure TPc1.

Preferably, the clamping pressure TPc1 is a rapidly increasing clamping pressure with rapid acquisition of blood pressure values up to SAP2ni+5 . . . SAP2ni+40 mmHg, preferably up to SAP2ni+20 mmHg. With the method in accordance with the invention, it is possible to obtain an orienting online determination of the fifth mean blood pressure value MAPni from the third mean blood pressure value MAP1ni and the fourth mean blood pressure value MAP2ni, and to obtain an orienting online determination of the third systolic blood pressure value SAPni from the first systolic blood pressure value SAP1ni and the second systolic blood pressure value SAP2ni or SAP2ni*. The following rates of increase are used:
a) increase to 0-30 mmHg during the first 1-2 s, from then
b) until the time of the fourth mean blood pressure value MAP2ni, for the determination of which a certain follow-up time is required, with 5-10 mmHg/pulse, preferably with 8 mmHg/pulse, from then
c) until the time of an upper clamping pressure limit, preferably SAP2ni+20 mmHg with 3-8 mmHg, preferably with 6 mmHg/pulse.

Then an immediate rough calculation of the third systolic blood pressure value SAPni (preferably weighted average of SAP1ni and SAP2ni), the fifth mean blood pressure value MAPni (preferably weighted average of MAP1ni and MAP2ni) and the third diastolic blood pressure value DAP1ni is carried out followed by the clamping pressure reduction.

The following speeds are used for clamping pressure reduction:
d) from an upper clamping pressure limit, preferably SAPni+20 mmHg to 90% DAP1ni, a constant decrease rate is set, so that between 10 and 50 tissue pressure pulse curves PKi, preferably 25 tissue pressure pulse curves PKi in the range between the systolic SAPni and 90% of the diastolic blood pressure value DAP1ni (=contains pulsatile blood pressure range between SAPni and DAP1ni) are detected.
e) followed by sudden reduction of clamping pressure, preferably after reaching 90% DAP1ni with simultaneous fine adjustment of SAPni, MAPni and DAP1ni.

The described method makes it possible to obtain various blood pressure values by means of a non-invasive measurement, which alone or in combination with other non-invasively determined blood pressure values lead to a reliable statement regarding the blood pressure values of a patient.

1. Method for non-invasively determining at least one blood pressure value (SAP1ni, MAP1ni, DAP1ni) from a tissue pressure signal (TP) using a pressure cuff (10) applied to an individual, the tissue pressure signal (TP) having a sequence of tissue pressure pulse curves (PKi), comprising:
identifying (S140) at least two individual tissue pressure pulse curves (PKI, PK2, . . . ) in the tissue pressure signal (TP);
determining (S150) at least one amplitude parameter (TPP) and one area parameter (TPA) for each identified tissue pressure pulse curve (PKi), the amplitude parameter (TPP) indicating the amplitude of the identified tissue pressure pulse curve (PKi) and the area parameter (TPA) indicating at least one partial area (TPA.top) enclosed by the tissue pressure pulse curve (PKi);
for each identified tissue pressure pulse curve (PKi), determining (S160) a pulsation power parameter (TPWP) indicating a shape of the tissue pressure pulse curve (PKi) based on at least the amplitude parameter (TPP) and the area parameter (TPA);
generating (S170) a parameter function (TPW-curve) which indicates a functional relationship between the determined pulsation power parameters (TPWP) of the tissue pressure pulse curves (PKi) and corresponding clamping pressures (TPc1) at the pressure cuff (10) or measuring times (t);
determining (S180-S195) at least one blood pressure value (SAP1ni, MAP1ni, DAP1ni) based on the parameter function (TPW-curve).

2. Method according to claim 1, wherein the blood pressure value (SAP1ni, MAP1ni, DAP1ni) is a systolic blood pressure value (SAP), a mean blood pressure value (MAP) and/or a diastolic blood pressure value (DAP).

3. Method according to claim 1 or 2, wherein the tissue pressure signal (TP) is determined over a pressure range of the pressure cuff (10) starting from a low clamping pressure to a high clamping pressure and/or from a high clamping pressure to a low clamping pressure or a section thereof.

4. Method according to claim 3, wherein the low clamping pressure is below the diastolic blood pressure value (DAP) and the high clamping pressure is above the systolic blood pressure value (SAP).

5. Method according to one of the preceding claims, wherein the area parameter (TPA) and the amplitude parameter (TPP) are preferably linked by multiplication of the amplitude parameter (TPP) and with the area parameter (TPA).

6. Method according to one of the preceding claims, wherein for each tissue pressure pulse curve (PKi) the pulsation power parameter (TPWP) is obtained by linking a potentiated area parameter (TPA) or/and a preferably triple potentiated amplitude parameter (TPP).

7. Method according to one of the preceding claims, wherein the area parameter (TPA) determined for each tissue pressure pulse curve (PKi) indicates a partial area (TPA.top, TPA+.top) which is enclosed by the tissue pressure pulse curve (PKi) and a straight line, preferably horizontally, intersecting the tissue pressure pulse curve (PKi) at a predetermined percentage amplitude value (x % TPP).

8. Method according to one of the preceding claims, wherein the amplitude parameter (TPP, TPP+) determined for each tissue pressure pulse curve (PKi) indicates a difference between a tissue pressure diastolic minimum (TPdia) and a tissue pressure systolic maximum (TPsys) or between the pressure value at which the clamping pressure is TPc1=0 and the tissue pressure systolic maximum (TPsys).

9. Method according to one of the preceding claims, wherein the parameter function (TPW-curve) is generated by assigning to each pulsation power parameter (TPWP) for the corresponding tissue pressure pulse curve (PKi) a measurement time (t(PKi)) or a clamping pressure (TPc1(PKi)).

10. Method according to one of the preceding claims, wherein a smoothing method or a curve fit is applied to the determined parameter function (TPW curve).

11. Method according to one of the preceding claims, wherein a first systolic blood pressure value (SAP1ni) is determined based on the parameter function (TPW curve) by the following:
a maximum parameter function value (TPW-curve.max) of the parameter function (TPW-curve) is determined;
in case of a pressure curve from a low to a high clamping pressure (TPc1), a first parameter function value (ax) following the maximum parameter function value (TPW-curve.max) and which has, with respect to the maximum parameter function value, a parameter function value reduced by a predetermined proportion and the corresponding first measuring time (t(ax)) or the corresponding first clamping pressure (TPc1(ax)) are determined; and
in case of a pressure curve from a high to a low clamping pressure (TPc1), a first parameter function value (ax) preceding the maximum parameter function value (TPW-curve.max) and which has, with respect to the maximum parameter function value, a parameter function value reduced by a predetermined proportion and the corresponding first measuring time (t(ax)) or the corresponding first clamping pressure (TPc1(ax)) are determined;
a first systolic blood pressure value (SAP1ni) corresponding to the first measuring time (t(ax)) or the first clamping pressure TPc1(ax) is determined from the tissue pressure signal (TP) or a signal dependent thereon, preferably an upper envelope (TPsys-curve) of the tissue pressure signal (UP).

12. Method according to one of the preceding claims, wherein an alternative first systolic blood pressure value (SAP1ni*, SAP1ni**, SAP4ni*, SAP4ni**) is determined based on the parameter function (TPW-curve) by the following:
a maximum parameter function value (TPW-curve.max) of the parameter function (TPW-curve) is determined;
a pressure value (TPc1@TPW-curve.max) corresponding to the clamping pressure (TPc1) at a time t(TPW-curve.max) of the maximum parameter function value (TPW-curve.max) or a pressure value corresponding to an upper envelope (TPsys-curve) of the tissue pressure signal (TP) at a time t(TPW-curve.max) of the maximum parameter function value (TPW-curve.max) is determined, and
a factor (TPc1%, TPc1+%) is applied to the pressure value (TPc1@TPW-curve.max) corresponding to the clamping pressure (TPc1) or a factor (TPsys-curve %, TPsys-curve+%) is applied to the pressure value (TPsys-curve@TPW-curve.max) corresponding to the upper envelope (TPsys-curve@TPW-curve.max) in order to determine an alternative first systolic blood pressure value (SAP1ni*, SAP Im**, SAP4ni*, SAP4ni**).

13. Method according to one of the preceding claims, wherein a first mean blood pressure value (MAP1Ani) is determined using the generated parameter function (TPW-curve) by the following:
a maximum parameter function value (TPW-curve.max) of the parameter function (TPW-curve) is determined;
in case of a pressure curve from a low to a high clamping pressure (TPc1), a second parameter function value (bx) preceding the maximum parameter function value (TPW-curve.max) and which has, with respect to the maximum parameter function value, a parameter function value reduced by a predetermined proportion and the corresponding second measuring time (t(bx)) or the corresponding second clamping pressure (TPc1(bx)) are determined;
in case of a pressure curve from a high to a low clamping pressure (TPc1), a second parameter function value (bx) following the maximum parameter function value (TPW-curve.max) and which has, with respect to the maximum parameter function value, a parameter function value reduced by a predetermined proportion and the corresponding second measuring time (t(bx)) or the corresponding second clamping pressure (TPc1(bx)) are determined; and
a first mean blood pressure value (MAP1Ani) corresponding to the second measuring time (t(bx)) or the second clamping pressure TPc1(bx) is determined from the tissue pressure signal (TP) or a signal dependent thereon, preferably the clamping pressure (TPc1).

14. Method according to one of the preceding claims, wherein a first diastolic blood pressure value (DAP1Ani) is determined using the generated parameter function (TPW curve) by the following:
a maximum parameter function value (TPW-curve.max) of the parameter function (TPW-curve) is determined;
in case of a pressure curve from a low to a high clamping pressure (TPc1), a third parameter function value (cx) preceding the maximum parameter function value (TPW-curve.max) and which has, with respect to the maximum parameter function value, a parameter function value reduced by a predetermined proportion and the corresponding third measuring time (t(cx)) or the corresponding third clamping pressure (TPc1(cx)) are determined;
in case of a pressure curve from a high to a low clamping pressure (TPc1), a third parameter function value (cx) following the maximum parameter function value (TPW-curve.max) and which has, with respect to the maximum parameter function value, a parameter function value reduced by a predetermined proportion and the corresponding third measuring time (t(cx)) or the corresponding third clamping pressure (TPc1(cx)) are determined; and
a first diastolic blood pressure value (MAP1Ani) corresponding to the third measuring time (t(cx)) or the third clamping pressure TPc1(cx) is determined from the tissue pressure signal (TP) or a signal dependent thereon, preferably the lower envelope (TPdia-curve) of the tissue pressure signal (TP).

15. Method according to one of the preceding claims, wherein a second diastolic blood pressure value (DAP1Bni) is determined from the first systolic blood pressure value (SAP1ni) and the first mean blood pressure value (MAPI Am) according to the estimation formula:
$$DAP 1 Bni = k 1 \cdot MAP 1 Ani - k 2 \cdot ( SAP 1 ni - MAPI Am ) - k 3 mmHg ,$$
$$wherein k 1 = ( 0.6 … 1 , 1 ) , k 2 = ( 0 , 15 … 0.4 ) and k 3 = ( - 5 … 5 ) .$$

16. Method according to claim 15, wherein the first diastolic blood pressure value (DAP1Ani) generated by means of the parameter function (TPW curve) and the second diastolic blood pressure value (DAP1Bni) determined from the estimation formula are each weighted in order to obtain a third averaged mean diastolic blood pressure value (DAP1ni).

17. Method according to one of the preceding claims, wherein a second mean blood pressure value (MAP1Bni) is obtained from the first systolic blood pressure value (SAP1ni) and the first diastolic blood pressure value (DAP1Ani) according to the estimation formula:
$$MAP 1 Bni = k 4 \cdot DAP 1 Ani + k5 \cdot ( SAP 1 ni - DAP 1 Ani ) - k 6 mmHg ,$$
$$wherein k 4 = ( 0.8 … 1.3 ) , k 5 = ( 0.25 … 0.5 ) , k 6 = ( - 5 … 5 ) .$$

18. Method according to claim 17, wherein the first mean blood pressure value (MAP1Ani) determined by means of the parameter function (TPW curve) and the second mean blood pressure value (MAP1Bni) determined from the estimation formula are each weighted in order to obtain a third average mean blood pressure value (MAP1ni).

19. Method according to one of the preceding claims, wherein further a second systolic blood pressure value (SAP2ni, SAP2ni*) is determined based on the detection of a systolic shape change of the tissue pressure pulse curves (PKi) during the systolic passage in a sequence of successive tissue pressure pulse curves (PKi).

20. A method for non-invasively determining a systolic blood pressure value (SAP2ni) from a tissue pressure signal (TP) using a pressure cuff applied to an individual, the tissue pressure signal (TP) having a sequence of tissue pressure pulse curves (PKi), comprising:
identifying (S240) at least two tissue pressure pulse curves (PKi) in the tissue pressure signal (TP);
determining (S250) at least one area ratio (TPA1.top/TPA2.top) based on two partial areas (TPA1.top, TPA2.top) each enclosed by a tissue pressure pulse curve (PKi);
determining (S260, S270, S280) a maximum change in the area ratio (TPA1.top/TPA2.top);
determining (S290) a measuring time (t) or clamping pressure (TPc1) at which the area ratio (TPA1.top/TPA2.top) has the maximum change,
wherein a pressure value corresponding to the determined measuring time (t) or clamping pressure (TPc1) is determined (S290) from the tissue pressure signal (TP) or a signal dependent thereon, which represents a second systolic blood pressure value (SAP2ni).

21. Method according to claim 20, further comprising:
determining (S260) a moving mean value ((TPA1.top/TPA2.top). mean) of the area ratio (TPA1.top/TPA2.top) over a number n of tissue pressure pulse curves (PKi),
determining (S270) a difference ((TPA1.top/TPA2.top).diff) of the moving mean value ((TPA1.top/TPA2.top).mean) of the area ratio and the individual area ratio ((TPA1.top/TPA2.top).t) for each of the n tissue pressure pulse curves (PKi),
generating (S280) a standard deviation function ((TPA1.top/TPA2.top).sd) from the differences (TPA1.top/TPA2.top.diff) for the n tissue pressure pulse curves (PKi) and determining a center of the half-width of a developing bell curve shape of the standard deviation function ((TPA1.top/TPA2.top).sd), and
determining (S290) the second systolic blood pressure value (SAP2ni) based on the tissue pressure signal (TP) or a signal dependent thereon at the center of the half-width.

22. Method according to claim 19, wherein the first systolic blood pressure value (SAP1ni) generated by means of the parameter function (TPW-curve) and the second systolic blood pressure value (SAP2ni, SAP2ni*) determined from a systolic shape change of the tissue pressure pulse curves (PKi) during the systolic passage are weighted and averaged to obtain a third mean systolic blood pressure value (SAPni).

23. Method according to one of the preceding claims, wherein a fourth average blood pressure value (MAP2ni) is determined by the following:
for successive tissue pressure pulse curves (PKi, PKi+1), an respective area (Areg) from a tissue pressure pulse curve (PKi) to the subsequent tissue pressure pulse curve (Pki+1) is calculated (S450);
the calculated area (Areg) is divided into two partial areas (Areg.sys, Areg.dia) and an area ratio (Areg.sys/Areg.dia) is determined (S460, S470); and
from a change in the area ratio (d(Areg.sys/Areg.dia)/dt or d(Areg.sys/Areg.dia)/dTPcl) of the successive tissue pressure pulse curves (PKi, PKi+1), a fourth mean blood pressure value (MAP2ni) is determined from a corresponding tissue pressure signal (TP) or a signal dependent thereon, preferably the clamping pressure (TPc1) (S480, S490).

24. A method for non-invasively determining a mean blood pressure value (MAP2ni) from a tissue pressure signal (TP) using a pressure cuff (10) applied to an individual, the tissue pressure signal (TP) having a sequence of tissue pressure pulse curves (PK1-PKn), comprising:
identifying (S440) individual tissue pressure pulse curves (PKi) in the tissue pressure signal (TP);
calculating (S450), for successive tissue pressure pulse curves (PKi), a respective area (Areg) from a tissue pressure pulse curve (PKi) to the subsequent tissue pressure pulse curve (PKi+1);
forming (S460) two partial areas (Areg.sys, Areg.dia) by dividing the calculated area (Areg); and
determining (S470) an area ratio (Areg.sys/Areg.dia) from the two partial areas (Areg.sys, Areg.dia); and
determining (S480) a maximum change in the area ratio (d(Areg.sys/Areg.dia)/dt or d(Areg.sys/Areg.dia)/dTPcl) in successive tissue pressure pulse curves (PKi, PKi+1);
determining (S490) a fourth mean blood pressure value (MAP2ni) on the basis of the tissue pressure signal (TP) or a signal dependent thereon, preferably the clamping pressure (TPc1), at the time of the maximum change in the area ratio (d(Areg.sys/Areg.dia)/dt).

25. Method according to claim 23, wherein a fifth weighted and averaged mean blood pressure value (MAPni) is determined from the third mean blood pressure value (MAP1ni) and the fourth mean blood pressure value (MAP2ni) by weighting and averaging.

26. Method according to any of the preceding claims, wherein the step (S140, S240, S340, S440) of identifying tissue pressure pulse curves (PKi) further comprises: extracting the tissue pressure pulse curves (PKi) by subtracting or filtering (S130, S230, S330, S430) at least one clamping pressure portion (TPc1) from the tissue pressure signal (TP) and/or identifying (S 100) at least two successive tissue pressure pulse curves (PKI, PK2, . . . ).

27. Method according to one of the preceding claims, wherein, during the measurement, a pressure range determined during the measurement is passed through with at least one predetermined or adaptive time-dependent pressure change rate.

28. A measuring device (90) for the non-invasive determination of blood pressure values from a tissue pressure signal (TP) detected on an individual by means of a pressure cuff (10), the measuring device (90) comprising at least one control unit (92) which is configured to carry out the method according to one of the preceding claims 1-27.

29. Measuring device (90) according to claim 28, further comprising a pressure transmitter (94) which is configured to build up and/or reduce a pressure in the pressure cuff (10) over a predetermined pressure range or a pressure range determined during the measurement.

30. A system for non-invasive blood pressure determination, comprising:
a pressure cuff (10) having at least one pressure sensor for detecting a tissue pressure signal (TP) on an individual; and
a measuring device (90) according to claim 27 and/or 28 for determining at least one blood pressure value from the detected tissue pressure signal (TP) or a signal derived therefrom.

31. System for non-invasive blood pressure determination according to claim 30, wherein the pressure cuff (10) is formed as a shell wrap cuff comprising an inner kink-resistant shell (30).
